(19) United States
(12) Patent Application Publication
(10) Pub. No.: US 2008/0269291 A1
(43) Pub. Date: Oct. 30, 2008

(54) Chemical Compounds

(76) Inventors: Jeffrey K. Kerns, King of Prussia, PA (US); Jakob Busch-Petersen, King of Prussia, PA (US); Huijie Li, King of Prussia, PA (US); Jeffrey Charles Boehm, King of Prussia, PA (US); Hong Nie, King of Prussia, PA (US); John J. Taggart, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/093,750

(22) PCT Filed: Nov. 17, 2006
(86) PCT No.: PCT/US06/61018

§ 371 Date: May 15, 2008

Related U.S. Application Data
(60) Provisional application No. 60/738,393, filed on Nov. 18, 2005.

(51) Int. Cl. A61K 31/454; C07D 401/04

(52) U.S. Cl. 514/323; 546/201

(57) ABSTRACT

The invention is directed to novel indolecarboxamide derivatives. Specifically, the invention is directed to compounds according to formula (I):

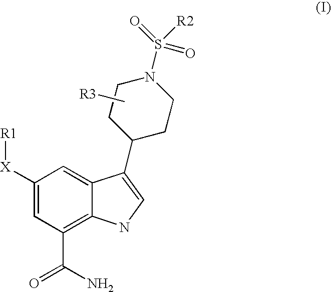

where R1, R2, R3 and X are defined below. These compounds are useful in the treatment of disorders associated with inappropriate IKK2 (also known as IKKβ) activity, in particular in the treatment and prevention of disorders mediated by IKK2 mechanisms including inflammatory and tissue repair disorders. Such disorders include rheumatoid arthritis, asthma, and COPD (chronic obstructive pulmonary disease).

### FIELD OF THE INVENTION

The invention is directed to certain indole carboxamide compounds, which are inhibitors of kinase activity. More specifically, the compounds are IKK2 inhibitors. These compounds are useful in the treatment of disorders associated with inappropriate IKK2 (also known as IKKβ) activity, in particular in the treatment and prevention of disorders mediated by IKK2 mechanisms including inflammatory and tissue repair disorders. Such disorders include rheumatoid arthritis, asthma, and COPD (chronic obstructive pulmonary disease).

### BACKGROUND OF THE INVENTION

An important large family of enzymes is the protein kinase enzyme family. Currently, there are about 500 different known protein kinases. However, because three to four percent of the human genome is a code for the formation of protein kinases, there may be many thousands of distinct and separate kinases in the human body. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify modulators of tyrosine kinases as well.

Nuclear factor κB (NF-κB) belongs to a family of closely related dimeric transcription factor complexes composed of various combinations of the Rel/NF-κB family of polypeptides. The family consists of five individual gene products in mammals, RelA(p65), NF-κB1 (p50/p105), NF-κB2 (p49/p100), c-Rel, and RelB, all of which can form hetero- or homodimers. These proteins share a highly homologous 300 amino acid “Rel homology domain” which contains the DNA binding and dimerization domains. At the extreme C-terminus of the Rel homology domain is a nuclear translocation sequence important in the transport of NF-κB from the cytoplasm to the nucleus. In addition, p65 and cRel possess potent transactivation domains at their C-terminal ends.

The activity of NF-κB is regulated by its interaction with a member of the inhibitor lκB family of proteins. This interaction effectively blocks the nuclear localization sequence on the NF-κB proteins, thus preventing migration of the dimer to the nucleus. A wide variety of stimuli activate NF-κB through what are likely to be multiple signal transduction pathways. Included are bacterial products (LPS), some viruses (HIV-1, HTLV-1), inflammatory cytokines (TNFα, IL-1), environmental and oxidative stress and DNA damaging agents. Apparently common to all stimuli however, is the phosphorylation and subsequent degradation of IκB. IκB is phosphorylated on two N-terminal serines by the recently identified IκB kinases (IKK-α and IKK-β). IKK-β is also known as IKK2. Site-directed mutagenesis studies indicate that these phosphorylations are critical for the subsequent activation of NF-κB in that once phosphorylated the protein is flagged for degradation via the ubiquitin-proteasome pathway. Free from IκB, the active NF-κB complexes are able to translocate to the nucleus where they bind in a selective manner to preferred gene-specific enhancer sequences. Included in the genes regulated by NF-κB are a number of cytokines and chemokines, cell adhesion molecules, acute phase proteins, immunoregulatory proteins, eicosanoid metabolizing enzymes and anti-apoptotic genes.

It is well-known that NF-κB plays a key role in the regulated expression of a large number of pro-inflammatory mediators including cytokines such as TNF, IL-1β, IL-6 and IL-8, cell adhesion molecules, such as ICAM and VCAM, and inducible nitric oxide synthase (iNOS). Such mediators are known to play a role in the recruitment of leukocytes at sites of inflammation and in the case of iNOS, may lead to organ destruction in some inflammatory and autoimmune diseases.

The importance of NF-κB in inflammatory disorders is further strengthened by studies of airway inflammation including asthma, in which NF-κB has been shown to be activated. This activation may underlie the increased cytokine production and leukocyte infiltration characteristic of these disorders. In addition, inhaled steroids are known to reduce airway hyperresponsiveness and suppress the inflammatory response in asthmatic airways. In light of the recent findings with regard to glucocorticoid inhibition of NF-κB, one may speculate that these effects are mediated through an inhibition of NF-κB.

Further evidence for a role of NF-κB in inflammatory disorders comes from studies of rheumatoid synovium. Although NF-κB is normally present as an inactive cytoplasmic complex, recent immunohistochemical studies have indicated that NF-κB is present in the nuclei, and hence active, in the cells comprising rheumatoid synovium. Furthermore, NF-κB has been shown to be activated in human synovial cells in response to stimulation with TNF-α or IL-1β. Such a distribution may be the underlying mechanism for the increased cytokine and eicosanoid production characteristic of this tissue. See Roshak, A. K., et al., J. Biol. Chem., 271, 31496-31501 (1996). Expression of IKK-β has been shown in synoviocytes of rheumatoid arthritis patients and gene transfer studies have demonstrated the central role of IKK-β in stimulated inflammatory mediator production in these cells. See Aupperele et al. J. Immunology 1999. 163:427-433 and Aupperle et al. J. Immunology 2001; 166:2705-11. More recently, the intra-articular administration of a wild type IKK-β adenoviral construct was shown to cause paw swelling while intra-articular administration of dominant-negative IKKβ inhibited adjuvant-induced arthritis in rat. See Tak et al. Arthritis and Rheumatism 2001, 44:1897-1907.

The NF-κB/Rel and IκB proteins are also likely to play a key role in neoplastic transformation and metastasis. Family members are associated with cell transformation in vitro and in vivo as a result of over expression, gene amplification, gene rearrangements or translocations. In addition, rearrangement and/or amplification of the genes encoding these proteins are seen in 20-25% of certain human lymphoid tumors. Further, NF-κB is activated by oncogenic ras, the most common defect in human tumors and blockade of NF-κB activation inhibits ras mediated cell transformation. In addition, a role for NF-κB in the regulation of apoptosis has been reported strengthening the role of this transcription factor in the regulation of tumor cell proliferation. TNF, ionizing radiation and DNA damaging agents have all been shown to activate NF-κB which in turn leads to the upregulated expression of several anti-apoptotic proteins. Conversely, inhibition of NF-κB has been shown to enhance apoptotic-killing by these agents in several tumor cell types. As this likely represents a major mechanism of tumor cell resistance to chemotherapy, inhibitors of NF-κB activation may be useful chemotherapeutic agents as either single agents or adjunct therapy. Recent reports have implicated NF-κB as an inhibitor of skeletal cell differentiation as well as a regulator of cytokine-induced muscle wasting (Guttridge et al. Science; 2000; 289: 2363-2365.) further supporting the potential of NFκB inhibitors as novel cancer therapies.

Several NF-κB inhibitors are described in C. Wahl, et al. J. Clin. Invest. 101 (5), 1163-1174 (1998), R. W. Sullivan, et al. J. Med. Chem. 41, 413-419 (1998), J. W. Pierce, et al. J. Biol. Chem. 272, 21096-21103 (1997).

The marine natural product hymenialdisine is known to inhibit NF-κB. Roshak, A., et al., JPET, 283, 955-961 (1997). Breton, J. J and Chabot-Fletcher, M. C., JPET, 282, 459-466 (1997).

Additionally, patent applications have been filed on aminothiophene inhibitors of the IKK2, see Callahan, et al., WO 2002030353; Baxter, et al., WO 2001058890, Faull, et al., WO 2003010158; Griffiths, et al., WO2003010163; Fancelli, et al., WO 200198290; imidazole inhibitors of IKK2, see Callahan, et al., WO 200230423; anilinophenylpyrimidine inhibitors of IKK2, see Kois, et al., WO 2002046171; β-carboline inhibitors of IKK2, see Ritzeler, et al., WO 2001068648, Ritzeler, et al., EP 1134221; Nielsch, et al. DE 19807993; Ritzeler, et al., EP 1209158; indole inhibitors of IKK2, see Ritzeler, et al., WO 2001030774; benzimidazole inhibitors of the IKK2, see Ritzeler, et al., DE 19928424; Ritzeler et al., WO 2001000610; aminopyridine inhibitors of IKK2, see Lowinger, et al., WO 2002024679; Murata, et al., WO 2002024693; Murata, et al., WO 2002044153; pyrazolaquinazoline inhibitors of IKK2, see Beaulieu, et al., WO 2002028860; Burke et al., WO 2002060386, Burke, et al. US 20030022898; quinoline inhibitors of IKK2, Browner, et al., WO2002041843, Browner, et al., US 20020161004 and pyridylcyanoguanidine inhibitors of IKK2, see Bjorkling, et al., WO 2002094813, Binderup et al, WO 2002094322 and Madsen, et al., WO 200294265 The natural products staurosporine, quercetin, K252a and K252b have been shown to be IKK2 inhibitors, see Peet, G. W. and Li, J. J. Biol. Chem., 274, 32655-32661 (1999) and Wisniewski, D., et al., Analytical Biochem. 274, 220-228 (1999). Synthetic inhibitors of IKK2 have also been described, see Burke, et al. J. Biol. Chem., 278, 1450-1456 (2003) and Murata, et al., Bioorg. Med. Chem. Lett., 13, 913-198 (2003) have described IKK2 inhibitors.

Thus, attempts have been made to prepare compounds that inhibit IKK2 activity and a number of such compounds have been disclosed in the art. However, in view of the number of pathological responses that are mediated by IKK2, there remains a continuing need for inhibitors of IKK2 which can be used in the treatment of a variety of conditions.

The present inventors have discovered novel indole carboxamide compounds, which are inhibitors of kinase activity, in particular inappropriate IKK2 activity. Such indole carboxamide derivatives are therefore useful in the treatment of disorders associated with inappropriate kinase, in particular inappropriate IKK2 activity in particular in the treatment and prevention of disease states mediated by IKK2 mechanisms including inflammatory and tissue repair disorders, particularly rheumatoid arthritis, inflammatory bowel disease, asthma and COPD (chronic obstructive pulmonary disease); osteoarthritis, osteoporosis and fibrotic diseases; dermatosis, including psoriasis, atopic dermatitis and ultraviolet radiation (UV)-induced skin damage; autoimmune diseases including systemic lupus eythematosus, multiple sclerosis, psoriatic arthritis, alkylosing spondylitis, tissue and organ rejection, Alzheimer's disease, stroke, atherosclerosis, restonosis, diabetes, glomerulonephritis, cancer, including Hodgkins disease, cachexia, inflammation associated with infection and certain viral infections, including acquired immune deficiency syndrome (AIDS), adult respiratory distress syndrome, and Ataxia Telangiestasia.

### SUMMARY OF THE INVENTION

The invention is directed to novel indole carboxamide derivatives. Specifically, the invention is directed to compounds according to formula (I):

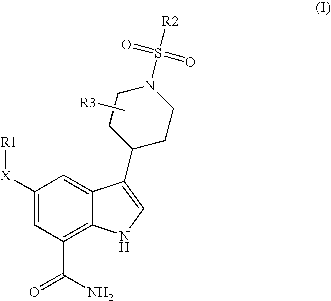

where X, R1, R2, and R3 are defined below.

### DETAILED DESCRIPTION OF THE INVENTION

The invention is directed to compounds according to formula (I):

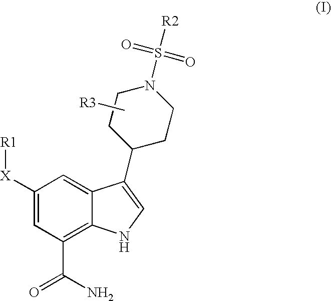

wherein:

### X is O, S, S(O), S(O)₂, —N(Rf), or —OC(O)O;

R1 is H, optionally substituted C₁-C₈ alkyl, C₁-C₆ haloalkyl, optionally substituted heterocycloalkyl, optionally substituted —C₁-C₃ alkylene-heterocycloalkyl, optionally substituted phenyl, optionally substituted —C₁-C₃ alkylene-phenyl, optionally substituted heteroaryl, or optionally substituted —C₁-C₃ alkylene-heteroaryl,

where said C₁-C₈ alkyl is optionally substituted with one substituent selected from the group consisting of: —NRfRf, —C(O)NRfRf, C₃-C₆ cycloalkyl, and C₁-C₆ alkoxy optionally substituted with one phenyl group;

where said heterocycloalkyl and —C₁-C₃ alkylene-heterocycloalkyl are optionally substituted with one to three substituents each independently selected from the group consisting of: halo, hydroxyl, oxo, and C₁-C₆ alkyl;

where said phenyl, —C₁-C₃ alkylene-phenyl, heteroaryl, and —C₁-C₃ alkylene-heteroaryl, are each optionally substituted with one to three substituents each independently selected from the group consisting of: halo, —CN, —N(Rb)SO₂Re, —N(Rb)C(O)Ra, —C(O)NRaRb, —C(O)NRfR9, —C(O)H, —SO₂Ri, —NRaRb, —SO₂NRaRb, —SO₂N RfRg, —ORc, —N(Rb)C(O)NRaRb, —N(Rb)C(O)N RfRg, —N(Rb)C(O)ORd, C₁-C₆ alkyl, C₁-C₆ alkyl substituted with one to three substituents independently selected from the group consisting of: —NRaRb, C₃-C₆ cycloalkyl, phenyl, —ORc, heterocycloalkyl, and heterocycloalkyl substituted with OH, —C(O)NH₂, or one or two C₁-C₆ alkyl groups; C₁-C₆ haloalkyl, C₁-C₆ haloalkyl substituted with one to three substituents each independently selected from the group consisting of —NRaRb, C₃-C₆ cycloalkyl, phenyl, heterocycloalkyl, and heterocycloalkyl substituted with one or two C₁-C₆ alkyl groups; heterocycloalkyl and heterocycloalkyl substituted with one or two C₁-C₆ alkyl groups;

R2 is optionally substituted C₁-C₆ alkyl, optionally substituted aryl, optionally substituted C₃-C₆ cycloalkyl, optionally substituted heteroaryl, or optionally substituted heterocycloalkyl,

wherein said C₁-C₆ alkyl is optionally substituted with one to three substituents each independently selected from the group consisting of: halo, —ORi, —NRgRh, —NHC(O)Rg, and Rj; and where said aryl and heteroaryl are optionally substituted with one to three substituents each independently selected from the following: halo, —ORg, nitro, cyano, CF₃, C₁-C₆ alkyl, C(O)R9, COORg, —NRgRh, —NHC(O)Rg, —C(O)NRgRh, —S(O)₂R9, —NHS(O)₂Rg, and —S(O)₂NRgRh; and where said C₃-C₆ cycloalkyl and heterocycloalkyl are optionally substituted by one to three substituents each independently selected from the group consisting of: —OH, oxo, C₁-C₆ alkyl, and C₁-C₆ haloalkyl;

R3 is one to three substituents each independently selected from the group consisting of: —OH, oxo, C₁-C₆ alkyl, and C₁-C₆ haloalkyl;
each Ra is independently selected from the group consisting of: H, optionally substituted C₁-C₃ alkyl, optionally substituted phenyl, optionally substituted heteroaryl, optionally substituted C₃-C₇ cycloalkyl, and optionally substituted heterocycloalkyl, where said C₁-C₃alkyl is optionally substituted with one to three substituents each independently selected from the group consisting of: halo, ORc, C₁-C₆ haloalkyl, phenyl, and heteroaryl; and where said phenyl, heteroaryl, C₃-C₇ cycloalkyl, and heterocycloalkyl are optionally substituted with one to three substituents each independently selected from the group consisting of: halo, ORc, C₁-C₆ alkyl, and C₁-C₆ haloalkyl;
each Rb is independently selected from the group consisting of: H and optionally substituted C₁-C₃ alkyl, where said C₁-C₃ alkyl is optionally substituted with one to three ORc groups;
each Rc is independently selected from the group consisting of: H, optionally substituted C₁-C₆ alkyl, optionally substituted C₁-C₆ haloalkyl, optionally substituted C₃-C₇ cycloalkyl, optionally substituted heterocycloalkyl, optionally substituted aryl, and optionally substituted heteroaryl, where said C₁-C₆ alkyl and C₁-C₆ haloalkyl are optionally substituted with one to three substituents each independently selected from the group consisting of: C₃-C₆ cycloalkyl, phenyl, heterocycloalkyl, and heteroaryl; and where said aryl and heteroaryl are optionally substituted with one to three substituents each independently selected from the group consisting of: halo, C₁-C₃ alkyl, C₁-C₃ haloalkyl and OH; and where said C₃-C₇ cycloalkyl and heterocycloalkyl are optionally substituted with one to three C₁-C₃ alkyl groups;
each Rd is independently optionally substituted C₁-C₃ alkyl, where said C₁-C₃ alkyl is optionally substituted with one to three substituents each independently selected from the group consisting of: C₃-C₆ cycloalkyl; phenyl optionally substituted with one to three substituents each independently selected from the group consisting of: halo, C₁-C₆ alkyl, and C₃-C₆ cycloalkyl; and heteroaryl optionally substituted with one to three substituents each independently selected from the group consisting of: halo, C₁-C₆ alkyl, and C₃-C₆ cycloalkyl;
each Re is independently selected from the group consisting of: optionally substituted C₁-C₆ alkyl, optionally substituted phenyl, optionally substituted heteroaryl, optionally substituted C₅-C₇ cycloalkyl, and optionally substituted heterocycloalkyl, where said C₁-C₆ alkyl is optionally substituted with one substituent selected from the group consisting of: ORc, trifluoromethyl, phenyl, heteroaryl, heterocycloalkyl optionally substituted with ORc or heterocycloalkyl, and NRaRb; where said phenyl and heteroaryl are optionally substituted with one to three substituents each independently selected from the group consisting of: halo, CN, C₁-C₆ alkyl, C₁-C₆ haloalkyl, N(Rb)C(O)Ra, and ORf; and where said C₅-C₇ cycloalkyl and heterocycloalkyl are optionally substituted with one to three substituents each independently selected from the group consisting of: halo, C₁-C₆ alkyl optionally substituted with ORc and C₃-C₆ cycloalkyl;
each Rf is independently selected from the group consisting of: H and C₁-C₆ alkyl;
each Rg is independently selected from the group consisting of: H, C₁-C₆ alkyl, C₃-C₆ cycloalkyl, heteroaryl, and phenyl;
each Rh is independently selected from the group consisting of: H and C₁-C₆ alkyl optionally substituted with one phenyl group;
each Ri is independently selected from the group consisting of: H, C₁-C₆ alkyl, C₁-C₆ haloalkyl, and phenyl; and
Rj is optionally substituted aryl, optionally substituted heteroaryl, optionally substituted C₃-C₆ cycloalkyl, or optionally substituted heterocycloalkyl,

where said aryl and heteroaryl are optionally substituted with one to three substituents each independently selected from the group consisting of: —ORf, nitro, cyano, CF₃, C₁-C₆ alkyl, C(O)Rf, COORf, —NRfRg, —NHC(O)Rf, —C(O)NRfRg, —S(O)₂Rf, —NHS(O)₂Rf, and —S(O)₂NRfRg; and where said C₃-C₆ cycloalkyl and heterocycloalkyl are optionally substituted with one to three substituents each independently selected from the group consisting of: —OH, oxo, C₁-C₆ alkyl, and C₁-C₆ haloalkyl.

In one embodiment X is O.

In one embodiment X is S.

In one embodiment X is S(O) or S(O)₂. In another embodiment X is S(O)₂.

In one embodiment X is N(Rf) where R(f) is H or CH₃.

In one embodiment X is OC(O)O.

In one embodiment R1 is H, optionally substituted C₁-C₈ alkyl, C₁-C₆ haloalkyl, optionally substituted heterocycloalkyl, optionally substituted —C₁-C₃ alkylene-heterocycloalkyl, optionally substituted phenyl, optionally substituted —C₁-C₃ alkylene-phenyl, optionally substituted naphthyl, optionally substituted —C₁-C₃ alkylene-naphthyl, optionally substituted heteroaryl, or optionally substituted —C₁-C₃ alkylene-heteroaryl,

where said C₁-C₈ alkyl is optionally substituted with one substituent selected from the group consisting of: cyano, —NRfRf, —C(O)NRfRf, C₃-C₆ cycloalkyl, and C₁-C₆ alkoxy optionally substituted with one phenyl group;

where said heterocycloalkyl and —C₁-C₃ alkylene-heterocycloalkyl are optionally substituted with one to three substituents each independently selected from the group consisting of: halo, hydroxyl, oxo, and C₁-C₆ alkyl;

where said phenyl, —C₁-C₃ alkylene-phenyl, heteroaryl, and —C₁-C₃ alkylene-heteroaryl, are each optionally substituted with one to three substituents each independently selected from the group consisting of: halo, —CN, —N(Rb)SO₂Re, —N(Rb)C(O)Ra, —C(O)NRaRb, —C(O)H, —SO₂R1, —NRaRb, —SO₂NRaRb, —ORc, —N(Rb)C(O)NRaRb, —N(Rb)C(O)ORd, —C(O)ORa, C₁-C₆ alkyl, C₁-C₆ alkyl substituted with one to three substituents independently selected from the group consisting of: —NRaRb, C₃-C₆ cycloalkyl, phenyl, —ORc, heterocycloalkyl, and heterocycloalkyl substituted with OH, —C(O)NH₂, or one or two C₁-C₆ alkyl groups; C₁-C₆ haloalkyl, C₁-C₆ haloalkyl substituted with one to three substituents each independently selected from the group consisting of —NRaRb, C₃-C₆ cycloalkyl, phenyl, heterocycloalkyl, and heterocycloalkyl substituted with one or two C₁-C₆ alkyl groups; heterocycloalkyl and heterocycloalkyl substituted with one or two C₁-C₆ alkyl groups;

In one embodiment R1 is H, optionally substituted C₁-C₈ alkyl, C₁-C₆ haloalkyl, optionally substituted phenyl, optionally substituted —C₁-C₃ alkylene-phenyl, and optionally substituted —C₁-C₃ alkylene-naphthyl,

where said C₁-C₈ alkyl is optionally substituted with one substituent selected from the group consisting of: cyano, —NRfRf, —C(O)NRfRf, C₃-C₆ cycloalkyl, and C₁-C₆ alkoxy optionally substituted with one phenyl group;

where said phenyl, —C₁-C₃ alkylene-phenyl, naphthyl, and —C₁-C₃ alkylene-naphthyl, are each optionally substituted with one to three substituents each independently selected from the group consisting of: halo, —CN, —C(O)NRaRb, —SO₂R1, —NRaRb, —ORc, —C(O)ORa, C₁-C₆ alkyl, C₁-C₆ alkyl substituted with one to three substituents independently selected from the group consisting of: —NRaRb, C₃-C₆ cycloalkyl, phenyl, —ORc, and C₁-C₆ haloalkyl.

In one embodiment R1 is H, unsubstituted —C₁-C₃ alkylene-phenyl, phenyl substituted with one or two substituents each independently selected from: halo, C₁-C₃ alkyl, NH—C(O)—CH₃, N(CH₂CH₃)₂ and methoxy; or C₁-C₈ alkyl optionally substituted with one N(CH₂CH₃)₂, C₃-C₆ cycloalkyl, halo, or C(O)NH₂ group.

In one embodiment R2 is C₁-C₆ alkyl optionally substituted with one to three substituents each independently selected from the group consisting of: halo, —ORi, —NRgRh, —NHC(O)Rg, and Rj. In another embodiment R2 is unsubstituted C₁-C₆ alkyl. In another embodiment R2 is ethyl.

In one embodiment each R3 group is H.

While embodiments for each variable have generally been listed above separately for each variable, compounds of this invention includes those in each variable in formula (I) may be independently selected from each described embodiment for each variable. Therefore, this invention is intended to include all combinations of embodiments for each variable.

Another embodiment of the present invention is a compound which is:
3-[1-(ethylsulfonyl)-4-piperidinyl]-5-hydroxy-1H-indole-7-carboxamide;
5-[(cyclopropylmethyl)oxy]-3-[1-(ethylsulfonyl)-4-piperidinyl]-1H-indole-7-carboxamide;
3-[1-(ethylsulfonyl)-4-piperidinyl]-5-(pentyloxy)-1H-indole-7-carboxamide;
3-[1-(ethylsulfonyl)-4-piperidinyl]-5-(octyloxy)-1H-indole-7-carboxamide;
3-[1-(ethylsulfonyl)-4-piperidinyl]-5-(heptyloxy)-1H-indole-7-carboxamide;
3-[1-(ethylsulfonyl)-4-piperidinyl]-5-[(2-phenylethyl)oxy]-1H-indole-7-carboxamide;
3-[1-(ethylsulfonyl)-4-piperidinyl]-5-[(3-phenylpropyl)oxy]-1H-indole-7-carboxamide;
5-[(2-chloroethyl)oxy]-3-[1-(ethylsulfonyl)-4-piperidinyl]-1H-indole-7-carboxamide;
3-[1-(ethylsulfonyl)-4-piperidinyl]-5-({4-[(phenylmethyl)oxy]butyl}oxy)-1H-indole-7-carboxamide;
3-[1-(ethylsulfonyl)-4-piperidinyl]-5-({2-[(phenylmethyl)oxy]ethyl}oxy)-1H-indole-7-carboxamide;
5-[(3-cyanopropyl)oxy]-3-[1-(ethylsulfonyl)-4-piperidinyl]-1H-indole-7-carboxamide;
5-[(4-amino-4-oxobutyl)oxy]-3-[1-(ethylsulfonyl)-4-piperidinyl]-1H-indole-7-carboxamide;
3-[1-(ethylsulfonyl)-4-piperidinyl]-5-[(phenylmethyl)oxy]-1H-indole-7-carboxamide; or
3-[1-(ethylsulfonyl)-4-piperidinyl]-5-[(phenylmethyl)oxy]-1H-indole-7-carboxamide.

Another embodiment of the invention is a compound which is:
5-{[(3,4-difluorophenyl)methyl]oxy}-3-[1-(ethylsulfonyl)-4-piperidinyl]-1H-indole-7-carboxamide;
5-{[(3-chlorophenyl)methyl]oxy}-3-[1-(ethylsulfonyl)-4-piperidinyl]-1H-indole-7-carboxamide;
methyl 4-[({7-(aminocarbonyl)-3-[1-(ethylsulfonyl)-4-piperidinyl]-1H-indol-5-yl}oxy)methyl]benzoate;
3-[1-(ethylsulfonyl)-4-piperidinyl]-5-{[(4-fluorophenyl)methyl]oxy}-1H-indole-7-carboxamide;
5-{[(3-cyanophenyl)methyl]oxy}-3-[1-(ethylsulfonyl)-4-piperidinyl]-1H-indole-7-carboxamide;
3-[1-(ethylsulfonyl)-4-piperidinyl]-5-[({2-[(phenylsulfonyl)methyl]phenyl}methyl)oxy]-1H-indole-7-carboxamide;
5-{[(2-cyanophenyl)methyl]oxy}-3-[1-(ethylsulfonyl)-4-piperidinyl]-1H-indole-7-carboxamide;
3-[1-(ethylsulfonyl)-4-piperidinyl]-5-[(2-naphthalenylmethyl)oxy]-1H-indole-7-carboxamide;
3-[1-(ethylsulfonyl)-4-piperidinyl]-5-[({3-[(trifluoromethyl)oxy]phenyl}methyl)oxy]-1H-indole-7-carboxamide;
3-[1-(ethylsulfonyl)-4-piperidinyl]-5-({[2-fluoro-4-(trifluoromethyl)phenyl]methyl}oxy)-1H-indole-7-carboxamide;
5-{[(3,5-difluorophenyl)methyl]oxy}-3-[1-(ethylsulfonyl)-4-piperidinyl]-1H-indole-7-carboxamide;
5-[({3-[(difluoromethyl)oxy]phenyl}methyl)oxy]-3-[1-(ethylsulfonyl)-4-piperidinyl]-1H-indole-7-carboxamide;
5-{[(3,4-dichlorophenyl)methyl]oxy}-3-[1-(ethylsulfonyl)-4-piperidinyl]-1H-indole-7-carboxamide;
5-{[(4-chlorophenyl)methyl]oxy}-3-[1-(ethylsulfonyl)-4-piperidinyl]-1H-indole-7-carboxamide;
3-[1-(ethylsulfonyl)-4-piperidinyl]-5-[(4-methylphenyl)oxy]-1H-indole-7-carboxamide;
3-[1-(ethylsulfonyl)-4-piperidinyl]-5-{[4-(methyloxy)phenyl]oxy}-1H-indole-7-carboxamide;
5-{[3-(diethylamino)phenyl]oxy}-3-[1-(ethylsulfonyl)-4-piperidinyl]-1H-indole-7-carboxamide;
3-[1-(ethylsulfonyl)-4-piperidinyl]-5-[(4-fluorophenyl)oxy]-1H-indole-7-carboxamide;
3-[1-(ethylsulfonyl)-4-piperidinyl]-5-[(2-methylpropyl)amino]-1H-indole-7-carboxamide;
3-[1-(ethylsulfonyl)-4-piperidinyl]-5-[methyl(phenyl)amino]-1H-indole-7-carboxamide;
3-[1-(ethylsulfonyl)piperidin-4-yl]-5-(phenylthio)-1H-indole-7-carboxamide;
5-[(4-chlorophenyl)thio]-3-[1-(ethylsulfonyl)-4-piperidinyl]-1H-indole-7-carboxamide;
5-[(2-chlorophenyl)thio]-3-[1-(ethylsulfonyl)-4-piperidinyl]-1H-indole-7-carboxamide;
3-[1-(ethylsulfonyl)-4-piperidinyl]-5-[(4-methylphenyl)thio]-1H-indole-7-carboxamide;
3-[1-(ethylsulfonyl)-4-piperidinyl]-5-[(4-fluorophenyl)thio]-1H-indole-7-carboxamide;
3-[1-(ethylsulfonyl)-4-piperidinyl]-5-[(3-fluorophenyl)thio]-1H-indole-7-carboxamide;
3-[1-(ethylsulfonyl)-4-piperidinyl]-5-[(3-fluorophenyl)thio]-1H-indole-7-carboxamide;
3-[1-(ethylsulfonyl)-4-piperidinyl]-5-[(2-fluorophenyl)thio]-1H-indole-7-carboxamide;
3-[1-(ethylsulfonyl)-4-piperidinyl]-5-[(3-methylphenyl)thio]-1H-indole-7-carboxamide;
5-{[2-(diethylamino)ethyl]thio}-3-[1-(ethylsulfonyl)-4-piperidinyl]-1H-indole-7-carboxamide;
5-[(2,4-dichlorophenyl)thio]-3-[1-(ethylsulfonyl)-4-piperidinyl]-1H-indole-7-carboxamide;
3-[1-(ethylsulfonyl)-4-piperidinyl]-5-[(2-methylpropyl)thio]-1H-indole-7-carboxamide;
5-{[4-(acetylamino)phenyl]thio}-3-[1-(ethylsulfonyl)-4-piperidinyl]-1H-indole-7-carboxamide;
3-[1-(ethylsulfonyl)-4-piperidinyl]-5-[(4-methylphenyl)sulfonyl]-1H-indole-7-carboxamide;
3-[1-(ethylsulfonyl)-4-piperidinyl]-5-[(3-fluorophenyl)sulfonyl]-1H-indole-7-carboxamide; or
5-{[4-(acetylamino)phenyl]sulfonyl}-3-[1-(ethylsulfonyl)-4-piperidinyl]-1H-indole-7-carboxamide.

### TERMS AND DEFINITIONS

“Alkyl” refers to a saturated hydrocarbon chain having the specified number of member atoms. For example, C₁-C₆ alkyl refers to an alkyl group having from 1 to 6 member atoms. Alkyl groups may be optionally substituted with one or more substituents as defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl.

“Alkylene” when used alone or in forming other groups (such as the C₁-C₆ alkylene-heteroaryl, C₁-C₆ alkylene-heterocycloalkyl, C₁-C₆ alkylene-C₄-C₇cycloalkyl, and C₁-C₆ alkylene-C₅-C₇cycloalkenyl groups) refers to a saturated divalent hydrocarbon chain having the specified number of member atoms. For example, C₁-C₆ alkylene refers to an alkylene group having from 1 to 6 member atoms. Alkylene groups when used alone may be optionally substituted with one or more substituents as defined herein. Alkylene groups when used to form other groups (such as the C₁-C₆ alkylene-heteroaryl, C₁-C₆ alkylene-heterocycloalkyl, C₁-C₆ alkylene-C₄-C₇cycloalkyl, and C₁-C₆ alkylene-C₅-C₇cycloalkenyl groups) are not substituted. For example, the group “optionally substituted C₁-C₆ alkylene-heteroaryl” contains only substituents on the heteroaryl group. Alkylene groups may be straight or branched. Representative branched alkylene groups have one, two, or three branches. Alkylene includes methylene, ethylene, propylene (n-propylene and isopropylene), butylene (n-butylene, isobutylene, and t-butylene), pentylene (n-pentylene, isopentylene, and neopentylene), and hexylene.

“Aryl” refers to an aromatic hydrocarbon ring. Aryl groups are monocyclic ring systems or bicyclic ring systems. Monocyclic aryl ring refers to phenyl. Bicyclic aryl rings refer to napthyl and rings wherein phenyl is fused to a cycloalkyl or cycloalkenyl ring having 5, 6, or 7 member atoms. Aryl groups may be optionally substituted with one or more substituents as defined herein.

“Cycloalkyl” refers to a saturated hydrocarbon ring having the specified number of member atoms. Cycloalkyl groups are monocyclic ring systems. For example, C₃-C₆ cycloalkyl refers to a cycloalkyl group having from 3 to 6 member atoms. Cycloalkyl groups may be optionally substituted with one or more substituents as defined herein. Cycloalkyl includes cyclopropyl, cyclobutyl, cyclopentyl, and cyclohexyl.

“Halo” refers to the halogen radical fluoro, chloro, bromo, or iodo.

“Haloalkyl” refers to an alkyl group wherein at least one hydrogen atom attached to a member atom within the alkyl group is replaced with halo. Haloalkyl includes trifluoromethyl.

“Heteroaryl” refers to an aromatic ring containing from 1 to 4 heteroatoms as member atoms in the ring. Heteroaryl groups containing more than one heteroatom may contain different heteroatoms. Heteroaryl groups may be optionally substituted with one or more substituents as defined herein. Heteroaryl groups are monocyclic ring systems or are fused, spiro, or bridged bicyclic ring systems. Monocyclic heteroaryl rings have 5 or 6 member atoms. Bicyclic heteroaryl rings have from 7 to 11 member atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, spiro, or bridged bicyclic ring system. Heteroaryl includes pyrrolyl, pyrazolyl, imidazolyl, oxazolyl, isoxazolyl, thiazolyl, isothiazolyl, furanyl, furazanyl, thienyl, triazolyl, pyridinyl, pyrimidinyl, pyridazinyl, pyrazinyl, triazinyl, tetrazinyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benopyranyl, benzoxazolyl, benzofuranyl, isobenzofuranyl, benzothiazolyl, benzothienyl, furopyridinyl, and napthyridinyl.

“Heteroatom” refers to a nitrogen, sulphur, or oxygen atom.

“Heterocycloalkyl” refers to a saturated or unsaturated ring containing from 1 to 4 heteroatoms as member atoms in the ring. However, heterocycloalkyl rings are not aromatic. Heterocycloalkyl groups containing more than one heteroatom may contain different heteroatoms. Heterocycloalkyl groups may be optionally substituted with one or more substituents as defined herein. Heterocycloalkyl groups are monocyclic ring systems having from 4 to 7 member atoms or a heterocycloalkyl group can be the bicyclic ring system decahydroisoquinoline. In certain embodiments, heterocycloalkyl is saturated. In other embodiments, heterocycloalkyl is unsaturated but not aromatic. Heterocycloalkyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, and azetidinyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, aryl, cycloalkyl, heterocycloalkyl, or heteroaryl, may be unsubstituted or substituted with one or more substituents as defined herein. “Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

g (grams); mg (milligrams);

L (liters); mL (milliliters);

μL (microliters); psi (pounds per square inch);

M (molar); mM (millimolar);

i.v. (intravenous); Hz (Hertz);

MHz (megahertz); mol (moles);

mmol (millimoles); rt (room temperature);

min (minutes); h (hours);

mp (melting point); TLC (thin layer chromatography);

Tᵣ (retention time); RP (reverse phase);

MeOH (methanol); i-PrOH (isopropanol);

TEA (triethylamine); TFA (trifluoroacetic acid);

TFAA (trifluoroacetic anhydride); THF (tetrahydrofuran);

DMSO (dimethylsulfoxide); AcOEt (ethyl acetate);

DME (1,2-dimethoxyethane); DCM (dichloromethane);

DCE (dichloroethane); DMF (N,N-dimethylformamide);

DMPU (N,N′-dimethylpropyleneurea); CDI (1,1-carbonyldiimidazole);

IBCF (isobutyl chloroformate); HOAc (acetic acid);

HOSu (N-hydroxysuccinimide); HOBT (1-hydroxybenzotriazole);

mCPBA (meta-chloroperbenzoic acid;

EDC (1-[3-dimethylamino) propyl]-3-ethylcarbodiimide hydrochloride);

BOC (tert-butyloxycarbonyl); FMOC (9-fluorenylmethoxycarbonyl);

DCC (dicyclohexylcarbodiimide); CBZ (benzyloxycarbonyl);

Ac (acetyl); atm (atmosphere);

TMSE (2-(trimethylsilyl)ethyl); TMS (trimethylsilyl);

TIPS (triisopropylsilyl); TBS (t-butyldimethylsilyl);

DMAP (4-dimethylaminopyridine); BSA (bovine serum albumin);

ATP (adenosine triphosphate); HRP (horseradish peroxidase);

DMEM (Dulbecco's modified Eagle medium);

HPLC (high pressure liquid chromatography);

BOP (bis(2-oxo-3-oxazolidinyl)phosphinic chloride);

TBAF (tetra-n-butylammonium fluoride);

H BTU(O-Benzotriazole-1-yl-N,N, N′,N′-tetramethyluroniumhexafluoro phosphate);

HEPES (4-(2-hydroxyethyl)-1-piperazine ethane sulfonic acid);

DPPA (diphenylphosphoryl azide);

fHNO₃ (fuming HNO₃);

EDTA (ethylenediaminetetraacetic acid);

TMEDA (N,N,N′,N′-tetramethyl-1,2-ethanediamine);

NBS (N-bromosuccinimide);

HATU (O-(7azabenzobenzotriazol-1-yl)-N,N,N′,N′-tetramethyluronium hexafluorophosphate);

DIPEA (diisopropylethylamine);

Imes (1,3-Bis(2,4,6-trimethylphenyl)imidazolium chloride);

dppf (1,1′-bis(diphenylphosphino)ferrocene);

CLR (Controlled Laboratory Reactor); and

NIS (N-iodsuccinimide).

All references to ether are to diethyl ether and brine refers to a saturated aqueous solution of NaCl.

The compounds according to formula I may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in formula I, or in any chemical structure illustrated herein, is not specified the structure is intended to encompass any stereoisomer and all mixtures thereof. Thus, compounds according to formula I containing one or more chiral center may be used as racemic mixtures, enantiomerically enriched mixtures, or as enantiomerically pure individual stereoisomers.

Individual stereoisomers of a compound according to formula I which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzamatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to formula I may also contain double bonds or other centers of geometric asymmetry. Where the stereochemistry of a center of geometric asymmetry present in formula I, or in any chemical structure illustrated herein, is not specified, the structure is intended to encompass the trans (E) geometric isomer, the cis (Z) geometric isomer, and all mixtures thereof. Likewise, all tautomeric forms are also included in formula I whether such tautomers exist in equilibrium or predominately in one form.

The skilled artisan will appreciate that pharmaceutically-acceptable salts of the compounds according to formula I may be prepared. Indeed, in certain embodiments of the invention, pharmaceutically-acceptable salts of the compounds according to formula I may be preferred over the respective free base or free acid because such salts impart greater stability or solubility to the molecule thereby facilitating formulation into a dosage form. Accordingly, the invention is further directed to pharmaceutically-acceptable salts of the compounds according to formula I.

As used herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to formula I may contain an acidic functional group. Suitable pharmaceutically-acceptable salts include salts of such acidic functional groups. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to formula I may contain a basic functional group and are therefore capable of forming pharmaceutically-acceptable acid addition salts by treatment with a suitable acid. Suitable acids include pharmaceutically-acceptable inorganic acids and pharmaceutically-acceptable organic acids. Representative pharmaceutically-acceptable acid addition salts include hydrochloride, hydrobromide, nitrate, methylnitrate, sulfate, bisulfate, sulfamate, phosphates acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

As used herein, the term “compounds of the invention” means both the compounds according to formula I and the pharmaceutically-acceptable salts thereof.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

### Compound Preparation

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Examples section.

Compounds of formula I can be prepared, for example, according to Scheme 1, depicted below:

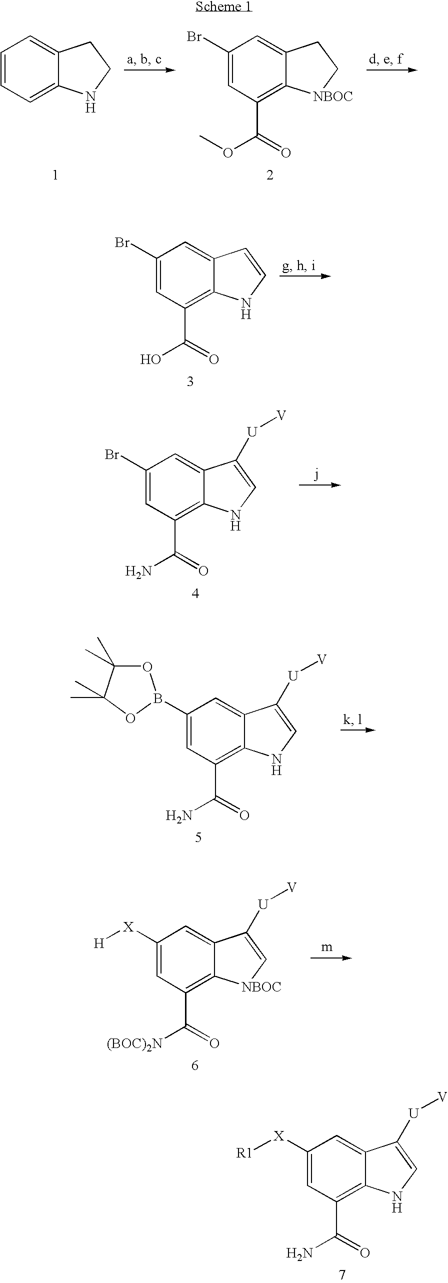

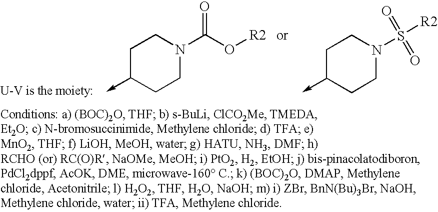

Scheme 1 represents a general scheme for the preparation of compounds according to formula I wherein X is O and R1 is an alkyl or substituted alkyl. Indoline 1 depicted as starting material is commercially available. Reaction conditions are as described above in the scheme; however, the skilled artisan will appreciate that certain modifications in the reaction conditions and/or reagents used are possible.

Treatment of indoline 1 with di-tertbutyl dicarbonate in a suitable solvent such as THF or methylene chloride produces the desired BOC protected product. Further transformation to the desired bromide 2 can be accomplished via lithiation using sec-butyllithium in the presence of TMEDA and quenching with methyl chloroformate followed by bromination with N-bromosuccinimide. Treatment of bromide 2 with trifluoroacetic acid followed by oxidation of the resulting indoline to the indole with manganese dioxide and subsequent hydrolysis of the methyl ester to the acid yields the desired carboxylic acid 3. Preparation of the primary carboxamide 4 can be completed via reaction of the carboxylic acid with ammonia in the presence of HATU. Incorporating the group U-V is performed via reaction with the appropriate aldehyde or ketone precursor to U-V. This transformation can be completed under either basic or acidic conditions. For the case where the group U-V is fully saturated, a subsequent reduction of the intermediate product will produce the title compound 4. As an example of such a reduction, for the case in Scheme 1 condition “i”, a hydrogenation reaction in the presence of PtO₂ completes the transformation to 4. In the case where U-V and/or XR1 contains a suitable protecting group, removal of the protecting group under the appropriate conditions and further transformation to other products may be accomplished. Subsequent transformation of the amine function of the group U-V to either the sulfonamide or amide of R2 can be performed with the appropriate sulfonyl or acid chloride or acid anhydride of R2. It will be appreciated by the skilled artisan that upon conversion to either the sulfonamide or amide of R2 the resulting product may require further elaboration to R2. This can include but is not limited to suitable protecting and functional group manipulations and reactions with amines/alcohols R5. Preparation of intermediate 5 and installation of the substituent XR1 can be accomplished via a transition metal mediated coupling using an appropriate catalyst and coupling partner. As an example of such a transformation, for the case in Scheme 1 condition “j”, a Suzuki cross-coupling reaction can be completed using bis-pinacolatoboron in the presence of PdCl₂dppf, AcOK, in DME. Subsequent protection of carboxamide and indole nitrogens using di-tert-butyldicarbonate and oxidation of the boronate to the phenol provides intermediate 6. This in turn can be converted to the title compound 7 via alkylation with the appropriate halide followed by deprotection. Alternatively, compounds of formula I can be prepared, for example, according to Scheme 2, depicted below:

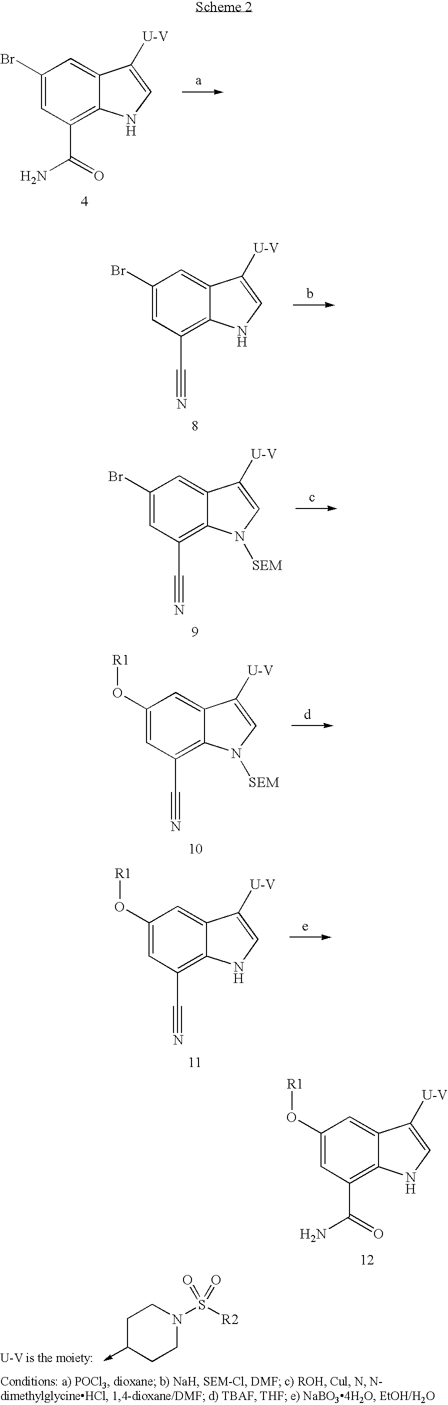

Scheme 2 represents a general scheme for the preparation of compounds according to formula I wherein R1 is a substituted phenyl group. Reaction conditions are as described above in the scheme; however, the skilled artisan will appreciate that certain modifications in the reaction conditions and/or the reagents used are possible.

Treatment of the amide 4 with POCl₃ in dioxane provides the nitrile product 8. Deprotonation of the indole N—H of 8 with NaH in DMF and treatment of the resulting anion with SEM-Cl gives the SEM-protected indole 9. Installation of the substituent OR1 can be accomplished in the presence of copper (I) iodide. As an example of such a transformation, for the case in Scheme 2 condition “c”, treatment of the intermediate 9 with a substituted phenol in the presence of CuI, Cs₂CO₃, and N,N-dimethylglycine.HCl in 1,4-dioxane/DMF provides intermediate 10. Subsequent deprotection of the indole nitrogen using TBAF provides intermediate 11. This in turn can be converted to the title compound 12 by hydrolysis of the nitrile to the carboxamide in the presence of either NaBO₃.

Alternatively, compounds of formula I can be prepared, for example, according to Scheme 3, depicted below:

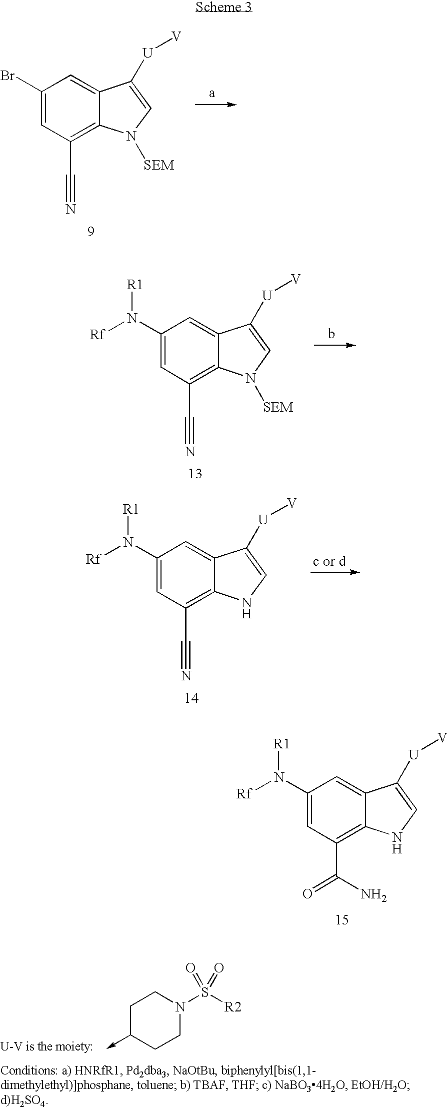

Scheme 3 represents a general scheme for the preparation of compounds according to formula I wherein Rf is an H or C₁-C₆ alkyl group, and R1 is an H, C₁-C₆ alkyl, C₃-C₆ cycloalkyl, heteroaryl, or phenyl group. Reaction conditions are as described above in the scheme; however, the skilled artisan will appreciate that certain modifications in the reaction conditions and/or the reagents used are possible.

Preparation of intermediate 13 and installation of the substituent NRfR1 can be accomplished via a transition metal mediated coupling using an appropriate catalyst and coupling partner. As an example of such a transformation, for the case in Scheme 2 condition “a”, a Buchwald-Hartwig cross-coupling reaction can be completed using HNRfRg in the presence of Pd₂ dba₃, 2-biphenylyl[bis(1,1-dimethylethyl)]phosphane, and NaOtBu in toluene. Subsequent deprotection of the indole nitrogen using TBAF provides intermediate 14. This in turn can be converted to the title compound 15 by hydrolysis of the nitrile to the carboxamide in the presence of either NaBO₃ or H₂SO₄.

Alternatively, compounds of formula I can be prepared, for example, according to Scheme 4, depicted below:

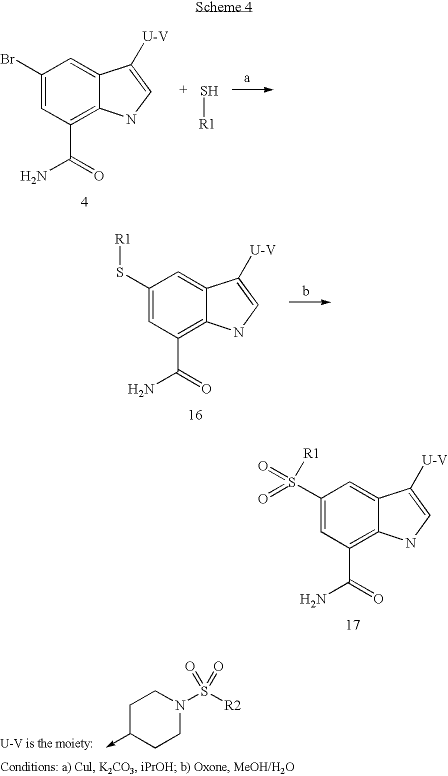

Scheme 4 represents a general scheme for the preparation of compounds according to formula I wherein R1 is a substituted alkyl, phenyl, or substituted aryl group. Reaction conditions are as described above in the scheme; however, the skilled artisan will appreciate that certain modifications in the reaction conditions and/or the reagents used are possible.

Treatment of intermediate 4 with a thiol in the presence of CuI, K₂CO₃, and i-PrOH provides the title compound 16, which in turn can be converted to the title compound 17 by oxidation of the sulfide in the presence of Oxone.

### Methods of Use

The compounds of the invention are inhibitors of IKK2. These compounds can be useful in the treatment of disorders wherein the underlying pathology is (at least in part) attributable to inappropriate IKK2 (also known as IKKβ) activity such as rheumatoid arthritis, inflammatory bowel disease, asthma, and COPD (chronic obstructive pulmonary disease). “Inappropriate IKK2 activity” refers to any IKK2 activity that deviates from the normal IKK2 activity expected in a particular patient. Inappropriate IKK2 activity may take the form of, for instance, an abnormal increase in activity, or an aberration in the timing and or control of IKK2 activity. Such inappropriate activity may result then, for example, from overexpression or mutation of the protein kinase leading to inappropriate or uncontrolled activation. Accordingly, in another aspect the invention is directed to methods of treating such disorders.

Such disorders include inflammatory and tissue repair disorders, particularly rheumatoid arthritis, inflammatory bowel disease, asthma and COPD (chronic obstructive pulmonary disease); osteoarthritis, osteoporosis and fibrotic diseases; dermatosis, including psoriasis, atopic dermatitis and ultraviolet radiation (UV)-induced skin damage; autoimmune diseases including systemic lupus eythematosus, multiple sclerosis, psoriatic arthritis, alkylosing spondylitis, tissue and organ rejection, Alzheimer's disease, stroke, atherosclerosis, restonosis, diabetes, glomerulonephritis, cancer, including Hodgkins disease, cachexia, inflammation associated with infection and certain viral infections, including acquired immune deficiency syndrome (AIDS), adult respiratory distress syndrome, and Ataxia Telangiestasia.

The methods of treatment of the invention comprise administering a safe and effective amount of a compound according to formula I or a pharmaceutically-acceptable salt thereof to a patient in need thereof. Individual embodiments of the invention include methods of treating any one of the above-mentioned disorders by administering a safe and effective amount of a compound according to formula I or a pharmaceutically-acceptable salt thereof to a patient in need thereof.

As used herein, “treat” in reference to a disorder means: (1) to ameliorate or prevent the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As indicated above, “treatment” of a disorder includes prevention of the disorder. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof.

As used herein, “safe and effective amount” in reference to a compound of the invention or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the disorder being treated, the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration range from 0.001 mg to 50 mg per kg of total body weight.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome or overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

The invention also provides a compound of the invention for use in medical therapy, and particularly in the treatment of disorders mediated by IKK2 activity. Thus, in a further aspect, the invention is directed to the use of a compound according to formula I or a pharmaceutically-acceptable salt thereof in the preparation of a medicament for the treatment of a disorder characterized by inappropriate IKK2 activity.

Particular disorders characterised by inappropriate IKK2 activity include inflammatory and tissue repair disorders, particularly rheumatoid arthritis, inflammatory bowel disease, asthma and COPD (chronic obstructive pulmonary disease); osteoarthritis, osteoporosis and fibrotic diseases; dermatosis, including psoriasis, atopic dermatitis and ultraviolet radiation (UV)-induced skin damage; autoimmune diseases including systemic lupus eythematosus, multiple sclerosis, psoriatic arthritis, alkylosing spondylitis, tissue and organ rejection, Alzheimer's disease, stroke, atherosclerosis, restenosis, diabetes, glomerulonephritis, cancer, including Hodgkins disease, cachexia, inflammation associated with infection and certain viral infections, including acquired immune deficiency syndrome (AIDS), adult respiratory distress syndrome, and Ataxia Telangiestasia as a result of inhibition of the protein kinase IKK2.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically-acceptable excipient. The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically may contain, for example, from 0.5 mg to 1 g, or from 1 mg to 700 mg, or from 5 mg to 100 mg of a compound of the invention.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when comingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of the invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds of the invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

In another aspect, the invention is directed to a liquid oral dosage form. Oral liquids such as solution, syrups and elixirs can be prepared in dosage unit form so that a given quantity contains a predetermined amount of a compound of the invention. Syrups can be prepared by dissolving the compound of the invention in a suitably flavored aqueous solution, while elixirs are prepared through the use of a non-toxic alcoholic vehicle. Suspensions can be formulated by dispersing the compound of the invention in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to a dosage form adapted for administration to a patient by inhalation. For example, the compound of the invention may be inhaled into the lungs as a dry powder, an aerosol, a suspension, or a solution.

Dry powder compositions for delivery to the lung by inhalation typically comprise a compound of the invention as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of the invention.

Aerosols may be formed by suspending or dissolving a compound of the invention in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of the invention will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

Suspensions and solutions comprising a compound of the invention may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of the invention may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of the invention. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions may be applied as a topical ointment or cream. When formulated in an ointment, the compound of the invention may be employed with either a paraffinic or a water-miscible ointment base. Alternatively, the compound of the invention may be formulated in a cream with an oil-in-water cream base or a water-in-oil base.

Pharmaceutical compositions adapted for nasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the compound of the invention.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

### PREPARATIONS AND EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Unless otherwise noted, all starting materials were obtained from commercial suppliers and used without further purification. Unless otherwise indicated, all temperatures are expressed in ° C. (degrees Centigrade). Unless otherwise indicated, all reactions are conducted under an inert atmosphere at room temperature. For reverse phase HPLC purification (unless otherwise stated), a 50×20 mm I.D. Luna C18 5μ column using acetonitrile containing 0.1% TFA and water containing 0.1% TFA and UV detection at 215 nM and 254 nM was used.

Nuclear magnetic resonance spectra were recorded at 400 MHz using a Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (8) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Mass spectra were taken on a PE Sciex Single Quadrupole LC/MS API-150 using electrospray (ES) ionization techniques. Elemental analyses were obtained using a Perkin-Elmer 240C elemental analyzer.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

### Intermediate 1: 1,1-dimethylethyl-2,3-dihydro-1H-indole-1-carboxylate

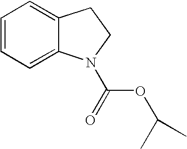

Indoline (10 g, 84 mmol) was dissolved in tetrahydrofuran (100 mL) and di-tert-butylcarbonate (22 g, 100 mmol) was added. The mixture was left stirring for 16 hours at room temperature under an inert nitrogen atmosphere. The tetrahydrofuran was removed in vacuo and the crude product purified by vacuum distillation to give 15.1 g (82%) of the title compound as a clear pale pink oil that crystallised upon standing (temperature: 160-162° C., pressure 1-0.1 mm Hg).

1H NMR (400 MHz, DMSO-D6) δ ppm 1.50 (s, 9H) 3.04 (t, J=8.7 Hz, 2H) 3.89 (t, J=8.8 Hz, 2H) 6.91 (td, J=7.3, 0.8 Hz, 1H) 7.13 (t, J=7.5 Hz, 1H) 7.18 (d, J=7.3 Hz, 1H) 7.5-7.8 (bs, 1H).

### Intermediate 2: 1-(1,1-dimethylethyl) 7-methyl-2,3-dihydro-1H-indole-1,7-dicarboxylate

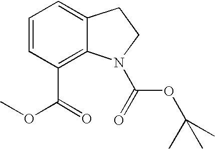

1,1-dimethylethyl 2,3-dihydro-1H-indole-1-carboxylate (5 g, 22.8 mmol) and N,N,N′,′-tetramethyl-1,2-ethanediamine (4.6 mL, 30.5 mmol) was dissolved in dry diethyl ether (300 mL) and cooled to −78° C. in an acetone/dry ice bath. Sec-butyl lithium (1.4 M solution in cyclohexane, 17.6 mL, 24.6 mmol) was added dropwise over 10 minutes and the reaction left stirring for 90 minutes at this temperature. Methyl chloroformate (8.8 mL, 10.8 g, 100 mmol) was added to the mixture and the reaction was allowed to warm up to room temperature over 1 hour. Water was added carefully to the mixture and the organic layer separated and washed 3 times with more water. The organic layer was dried over magnesium sulfate, filtered and concentrated in vacuo to give 4.91 g (78%) of the title compound as a gummy yellow solid.

1H NMR (400 MHz, DMSO-D6) δ ppm 1.44 (s, 9H) 3.06 (t, J=8.2 Hz, 2H) 3.69 (s, 3H) 4.02 (t, J=8.3 Hz, 2H) 7.06 (t, J=7.5 Hz, 1H) 7.35 (d, J=7.5 Hz, 1H) 7.39 (dd, J=7.4, 1.1 Hz, 1H) MS m/z 278 (M+1)⁺ Rt 3.18 min.

### Intermediate 3: 1-(1,1-dimethylethyl)-7-methyl-5-bromo-2,3-dihydro-1H-indole-1,7-dicarboxylate

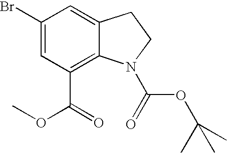

1-(1,1-dimethylethyl) 7-methyl 2,3-dihydro-1H-indole-1,7-dicarboxylate (3.1 g, 11.2 mmol) and N-bromosuccinimide (2.0 g, 11.2 mmol) were dissolved in dry dichloromethane (100 mL) and stirred under a nitrogen atmosphere at room temperature for 16 hours. The reaction was partitioned with sodium hydroxide solution (2 M), separated and washed with more sodium hydroxide solution. The organic layer was dried over magnesium sulfate and concentrated in vacuo to give 3.55 g (89%) of the title compound as a gummy red solid.

1H NMR (400 MHz, DMSO-D6) δ ppm 1.41 (s, 9H) 3.09 (t, J=8.3 Hz, 2H) 3.70 (s, 3H) 4.02 (t, J=8.3 Hz, 2H) 7.46 (s, 1H) 7.60 (s, 1H); MS m/z 356/358 (1:1 ratio) (M+1)⁺ Rt 3.52 min.

### Intermediate 4: Methyl 5-bromo-2,3-dihydro-1H-indole-7-carboxylate

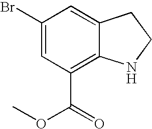

1-(1,1-dimethylethyl) 7-methyl 5-bromo-2,3-dihydro-1H-indole-1,7-dicarboxylate (9 g, 25 mmol) was dissolved in trifluoroacetic acid (6 mL) and stirred at room temperature for 16 hours. Dichloromethane and sodium hydroxide solution (2 M) were added and the organic layer washed twice with sodium hydroxide solution until the aqueous layer pH>7. The organic layer was then concentrated in vacuo to give 6.5 g (100%) of the title compound as a brown solid.

1H NMR (400 MHz, DMSO-D6) δ ppm 2.99 (t, J=8.5 Hz, 2H) 3.61 (t, J=8.4 Hz, 2H) 3.78 (s, 3H) 6.72 (s, 1H) 7.28 (d, J=1 Hz, 1H) 7.46 (d, J=2 Hz, 1H); MS m/z 256/258 (1:1 ratio) (M+1)⁺ Rt 3.32 min.

### Intermediate 5: Methyl 5-bromo-1H-indole-7-carboxylate

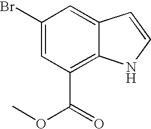

Methyl 5-bromo-2,3-dihydro-1H-indole-7-carboxylate (6.5 g, 25 mmol) was dissolved in tetrahydrofuran (100 mL). Activated manganese dioxide (5 μm particle size, 22 g, 250 mmol) was added and the mixture stirred at room temperature for 16 hours. A further 22 g of activated manganese dioxide was added and the reaction stirred for 96 hours. The reaction was then filtered through celite and concentrated in vacuo to give 5.1 g (80%) of the title compound as a beige solid.

1H NMR (400 MHz, DMSO-D6) δ ppm 3.94 (s, 3H) 6.58 (d, J=3 Hz, 1H) 7.48 (d, J=3 Hz, 1H) 7.8 (d, J=2 Hz, 1H) 8.07 (d, J=1.8 Hz, 1H) 11.39 (bs, 1H); MS m/z 252/254 (1:1 ratio) (M−1) Rt 3.41 min.

### Intermediate 6: 5-bromo-1H-indole-7-carboxylic acid

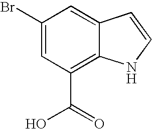

Methyl 5-bromo-1H-indole-7-carboxylate (5 g, 19.7 mmol) was dissolved in methanol (200 mL) and a solution of lithium hydroxide (0.99 g, 41 mmol) in water (10 mL) was added. The mixture was heated at reflux for 50 hours. The methanol was removed in vacuo and the residue diluted with aqueous hydrochloric acid (2 M). The resulting precipitate was filtered off and dried in a heated vacuum pistol to give 4.7 g (99%) of the title compound as a beige solid.

1H NMR (400 MHz, DMSO-D6) δ ppm 6.54 (dd, J=2.0, 3.2 Hz, 1H) 7.42 (t, J=2.8 Hz, 1H) 7.77 (d, J=2 Hz, 1H) 8.03 (d, J=1.8 Hz, 1H) 11.27 (s, 1H) 13.1-13.7 (bs, 1H) MS m/z 238/240 (1:1 ratio) (M−1) Rt 3.41 min.

### Intermediate 7: 5-bromo-1H-indole-7-carboxamide

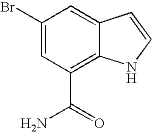

To a solution of 5-bromo-1H-indole-7-carboxylic acid (10.0 g, 42 mmol) in CH₂Cl₂ (100 mL) at room temperature, EDC (9.66 g, 50.4 mmol), HOBt (6.81 g, 50.4 mmol) and NH₃ (2.0 M in MeOH, 84 mL, 168 mmol) were added. The reaction mixture was stirred at room temperature for 16 hours. The solvent was evaporated and the residue partitioned between ethyl acetate (100 mL) and water (100 mL). The water layer was extracted with ethyl acetate (2×100 mL) and the combined organic phase was dried over MgSO₄ and concentrated to give 10 g (98%) of the title compound as a crude product. This crude product was used directly in the next step without further purification.

LC/MS: m/z 240.0 (M+H) Rt 1.95 min.

### Intermediate 8: 1,1-dimethylethyl-4-[7-(aminocarbonyl)-5-bromo-1H-indol-3-yl]-3,6-dihydro-1(2H)-pyridinecarboxylate

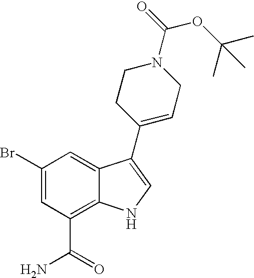

To a solution of 5-bromo-1H-indole-7-carboxamide (10 g, 41.8 mmol) in methanol (5 mL), 1,1-dimethylethyl 4-oxo-1-piperidinecarboxylate (684 mg, 3.42 mmol) and sodium methoxide (0.5 M in THF, 13.7 mL, 6.8 mmol) were added. The reaction mixture was stirred at reflux temperature for 16 hours. All solvent was evaporated under reduced pressure. The residue was partitioned between ethyl acetate (100 mL) and water (100 mL). The combined organic phase was dried over MgSO₄ and concentrated under reduced pressure, and purified by flash column chromatography (ethyl acetate/hexane, 1/1) to give 7.4 g (43%) of the title compound.

LC/MS: m/z 420.0 (M+H) Rt 2.35 min.

### Intermediate 9: 1,1-dimethylethyl-4-[7-(aminocarbonyl)-5-bromo-1H-indol-3-yl]-1-piperidine carboxylate

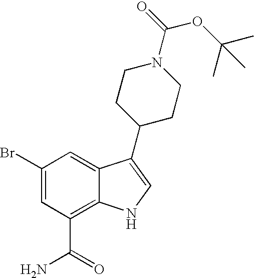

To a solution of 1,1-dimethylethyl 4-[7-(aminocarbonyl)-5-bromo-1H-indol-3-yl]-3,6-dihydro-1(2H)-pyridinecarboxylate (7.41 g, 17.6 mmol) in ethanol (600 mL), platinum oxide (200 mg, 5%) was added. The reaction mixture was hydrogenated under an atmosphere of H₂ balloon for 16 hours. The resulting mixture was filtered through celite and the filtrate was concentrated. The resulting residue was purified by flash column chromatography (Ethyl acetate/hexanes, 1:4 to 2:1 v/v) to give 3.6 g (48%) of the title compound.

LC/MS: m/z 422.0 (M+H) Rt 2.25 min.

### Intermediate 10: 5-bromo-3-(4-piperidinyl)-1H-indole-7-carboxamide

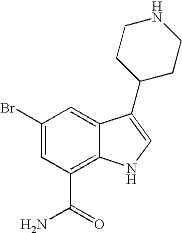

To a solution of 1,1-dimethylethyl 4-[7-(aminocarbonyl)-5-bromo-1H-indol-3-yl]-1-piperidinecarboxylate (1.56 g, 3.7 mmol) in methanol (10 mL), HCl in dioxane (4M, 35.5 mL) was added. The reaction mixture was stirred at room temperature for 2 hours. The solvent was evaporated under reduced pressure and the resulting residue was partitioned between ethyl acetate (50 mL) and 5% aqueous NaOH (50 mL). The aqueous layer was washed with ethyl acetate (2×50 mL) and the combined organic phases were dried and concentrated under reduced pressure to give 685 mg (58%) of the title compound, which was used in the next step without further purification.

LC/MS: m/z 322.0 (M+H) Rt 1.45 min.

### Intermediate 11: 5-bromo-3-[1-(ethanesulfonyl)-4-piperidinyl]-1H-indole-7-carboxamide

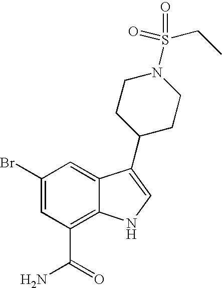

To 5-bromo-3-(4-piperidinyl)-1H-indole-7-carboxamide (900 mg, 2.8 mmol) in CH₂Cl₂ (100 mL) at 0° C., ethanesulfonyl chloride (0.8 mg, 8.4 mmol) and triethylamine (1.6 mL, 11.2 mmol) were added. The reaction mixture was stirred at 0° C. for 30 min. after which time the mixture was partitioned between CH₂Cl₂ and water. The aqueous phase was extracted with CH₂Cl₂ (2×50 mL) and the combined organic phase dried over MgSO₄ and concentrated under reduced pressure. The resulting residue was purified by solid phase extraction on a 500 mg aminopropyl column (International Sorbent Technologies) eluting with chloroform (2×30 mL) and ethyl acetate (50 mL) to give 800 mg, (69%) of the title compound.

LC/MS: m/z 414.0 (M+H) Rt 2.2 min.

### Intermediate 12: 3-[1-(ethylsulfonyl)-4-piperidinyl]-5-(4,4,5,5-tetramethyl-1,3,2-dioxaborolan-2-yl)-1H-indole-7-carboxamide

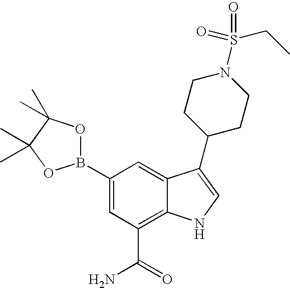

To 5-bromo-3-[1-(ethylsulfonyl)-4-piperidinyl]-1H-indole-7-carboxamide (1.0 g, 2.42 mmol) in DME (18 mL), bis(pinacolato)diboron (1.84 g, 7.3 mmol), potassium acetate (1.43 g, 14.5 mmol) and PdCl₂(dppf) (141 mg, 0.19 mmol) were added. The reaction mixture was heated by microwave at 150° C., 90 W for 3 hours. All solvent was removed in vacuo, followed by extracted with water (100 mL) and ethyl acetate (100 mL). The water layer was extracted with ethyl acetate (2×100 mL). The combined organic layers were extracted with brine (100 mL), dried over anhydrous magnesium sulfate, and concentrated. The crude product was washed with methylene chloride (20 mL) to give 800 mg (72%) of the title compound, which was carried on to next step without further purification.

LC/MS: m/z 462.4 (M+H) Rt 2.26 min.

### Intermediate 13: 1,1-dimethylethyl 7-[(bis{[(1,1-dimethylethyl)oxy]carbonyl}amino)carbonyl]-3-[1-(ethylsulfonyl)-4-piperidinyl]-5-(4,4,5,5-tetramethyl-1,3,2-dioxaborolan-2-yl)-1H-indole-1-carboxylate

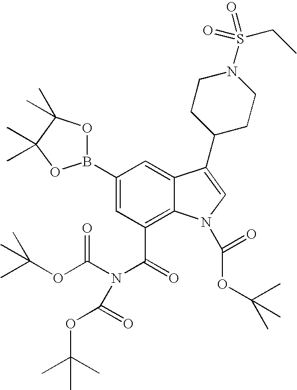

3-[1-(ethylsulfonyl)-4-piperidinyl]-5-(4,4,5,5-tetramethyl-1,3,2-dioxaborolan-2-yl)-1H-indole-7-carboxamide (100 mg, 0.217 mmol) in CH₂Cl₂ (2.5 mL) and CH₃CN (2.5 mL) was treated with stirring with BOC anhydride (156 mg, 0.716 mmol) followed by dimethylaminopyridine (DMAP) (8.5 mg, 0.07 mmol). The brown mixture slowly formed a solution which was stirred overnight at room temperature. The reaction was stripped to dryness, and the residue was taken up in a small amount of EtOAc-hexane (9:1) and very quickly passed down a silica gel column (10 g/60 mL) to give 123 mg (74%) of the title compound.

LC/MS: m/z 462.4 (M+H—loss of 3 Boc groups) Rt 2.95 min.

### Intermediate 14: 1,1-dimethylethyl 7-[(bis{[(1,1-dimethylethyl)oxy]carbonyl}amino)carbonyl]-3-[1-(ethylsulfonyl)-4-piperidinyl]-5-hydroxy-1H-indole-1-carboxylate

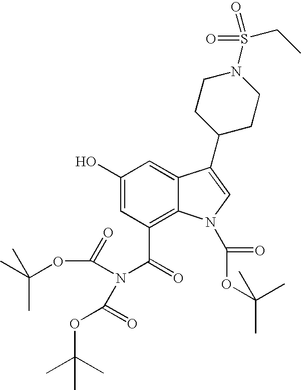

NaOH (1.2 mg, 0.03 mmol) was added to a solution of 1,1-dimethylethyl 7-[(bis{[(1,1-dimethylethyl)oxy]carbonyl}amino)carbonyl]-3-[1-(ethylsulfonyl)-4-piperidinyl]-5-(4,4,5,5-tetramethyl-1,3,2-dioxaborolan-2-yl)-1H-indole-1-carboxylate (25 mg, 0.033 mmol) in 1,2-dimethoxyethane (1.0 mL). A 30% solution of H₂O₂ (0.0036 mL, 3.6 eq.) was added, and the reaction was stirred for 2.5 h at room temperature. The solvent was removed and the resulting residue was taken up in EtOAc and H₂O and acidified with a few crystals of citric acid with rapid stirring. The layers were separated and the organic layer washed with saturated aqueous NaCl, dried (Na₂SO₄), and evaporated to give 23 mg. (quant.) of the title compound as a nearly colorless residue.

LC/MS=m/z 352 [M+H]⁺ (loss of three BOC groups) Rt 2.52 min.

### Intermediate 15: 1,1-dimethylethyl 7-[(bis{[(1,1-dimethylethyl)oxy]carbonyl}amino)carbonyl]-3-[1-(ethylsulfonyl)-4-piperidinyl]-5-[(phenylmethyl)oxy]-1H-indole-1-carboxylate

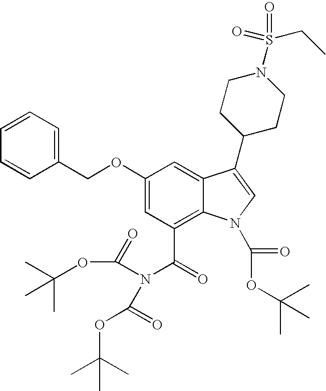

A solution of 1,1-dimethylethyl 7-[(bis{[(1,1-dimethylethyl)oxy]carbonyl}amino)carbonyl]-3-[1-(ethylsulfonyl)-4-piperidinyl]-5-hydroxy-1H-indole-1-carboxylate (90 mg, 0.138 mmol) in acetone (3.0 mL) was treated with benzyl bromide (52 μL, 75 mg, 0.44 mmol) and K₂CO₃ (66 mg, 0.48 mmol) with stirring. The mixture was stirred overnight at room temperature. The reaction was evaporated to give 110 mg (>95%) of the title compound, which was carried on to the next step without further purification.

LC/MS=m/z 742.8 [M+H]⁺ (loss of three BOC groups) Rt 2.49 min.

### Intermediate 16: 5-bromo-3-[1-(ethylsulfonyl)-4-piperidinyl]-1H-indole-7-carbonitrile

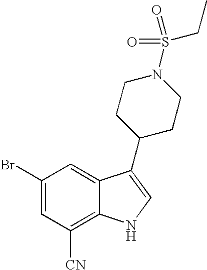

To 5-bromo-3-[1-(ethanesulfonyl)-4-piperidinyl]-1H-indole-7-carboxamide (2.10 g, 5.0 mmol) in dioxane (40 mL) was added POCl₃ (5.0 mL) at room temperature. The reaction mixture was heated at 45° C. overnight. The reaction was concentrated and treated with EtOAc and water. The water layer was extracted once using EtOAc. The combined organic layers were dried with MgSO₄, and concentrated to give 1.70 g (85%) of the title compound, which was carried on to the next step without further purification.

LC/MS: m/z 397 (M+H) Rt 2.23 min

### Intermediate 17: 5-bromo-3-[1-(ethylsulfonyl)-4-piperidinyl]-1-({[2-(trimethylsilyl)ethyl]oxy}methyl)-1H-indole-7-carbonitrile

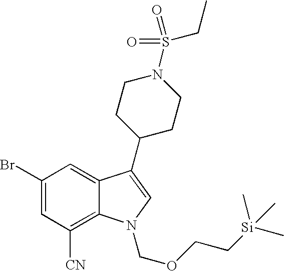

NaH (17 mg, 0.69 mmol) was added to a solution of 5-bromo-3-[1-(ethylsulfonyl)-4-piperidinyl]-1/H-indole-7-carbonitrile (182 mg, 0.46 mmol) in DMF (10 mL) at room temperature. After stirring 15 min, {2-[(chloromethyl)oxy]ethyl}(trimethyl)silane (0.10 mL, 0.55 mmol) was added. The reaction was kept at room temperature overnight, followed by addition of EtOAc and water. The water layer was extracted once with EtOAc and the combined organic layers were washed with brine. The organic layers were then dried using MgSO₄ and concentrated. The concentrated residue was purified using flash chromatography eluting with 30% EtOAc in hexane to give 160 mg (66%) of the title compound.

¹H NMR (400 MHz, CDCl₃) δ 7.93 (d, 1H), 7.66 (2, 1H), 7.07 (s, 1H), 5.72 (s, 2H), 3.97 (t, 2H), 3.57 (t, 2H), 2.87-3.06 (m, 5H), 2.08 (t, 2H), 1.76-1.85 (m, 2H), 1.42 (t, 3H), 1.28 (t, 2H), 0.00 (s, 9H)

### Intermediate 18: 3-[1-(ethylsulfonyl)-4-piperidinyl]-5-[(4-methylphenyl)oxy]-[1-([2-(trimethylsilyl)ethyl]oxy]methyl)-1H-indole-7-carbonitrile

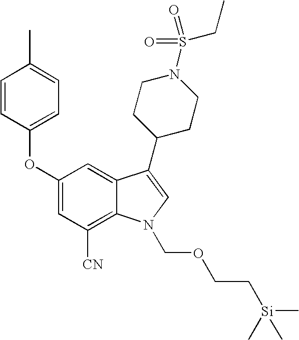

To a microwave vessel was added 5-bromo-3-[1-(ethylsulfonyl)-4-piperidinyl]-1-({[2-(trimethylsilyl)ethyl]oxy}methyl)-1H-indole-7-carbonitrile (100 mg, 0.2 mmol), 4-methylphenol (40 mg, 0.4 mmol), CuI (20 mg, 0.1 mmol), N,N-dimethylglycine.HCl (16 mg, 0.1 mmol), Cs₂CO₃ (128 mg, 0.4 mmol), dioxane (2 mL) and DMF (0.5 mL). The reaction was run in the microwave at 160° C. for a total of 60 min. The solvent was evaporated and EtOAc and water were added. The water layer was extracted once with EtOAc and the combined organic layers were washed with brine. The organic layers were then dried over MgSO₄ and concentrated. The crude product was purified by flash chromatography eluting with 2:1 hexanes/EtOAc to give 50 mg (49%) of the title compound.

LC/MS: m/z 527 (M+H) Rt 3.03 min

### Intermediate 19: 3-[1-(ethylsulfonyl)-4-piperidinyl]-5-[(4-methylphenyl)oxy]-1H-indole-7-carbonitrile

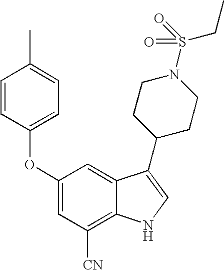

TBAF (0.40 mL, 0.40 mmol) was added to the solution of 3-[1-(ethylsulfonyl)-4-piperidinyl]-5-[(4-methylphenyl)oxy]-1-({[2-(trimethylsilyl)ethyl]oxy}methyl)-1H-indole-7-carbonitrile (50 mg, 0.089 mmol) in THF (3 mL). The reaction was maintained overnight at 65° C., followed by addition of EtOAc and water. It was then extracted once using EtOAc and dried over (MgSO₄). The organic layers were dried and concentrated to give 38 mg (100%) of crude product that was of sufficient purity to carry on to the next step.

LC/MS: m/z 424 (M+H) Rt 2.44 min

### Intermediate 20: 3-[1-(ethylsulfonyl)-4-piperidinyl]-5-[(4-methoxyphenyl)oxy]-1-({[2-(trimethylsilyl)ethyl]oxy}methyl)-1H-indole-7-carbonitrile

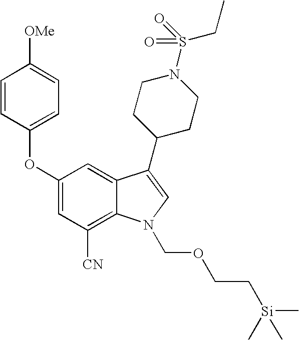

To a microwave vessel was added 5-bromo-3-[1-(ethylsulfonyl)-4-piperidinyl]-1-({[2-(trimethylsilyl)ethyl]oxy}methyl)-1H-indole-7-carbonitrile (100 mg, 0.2 mmol), 4-methoxyphenol (47 mg, 0.4 mmol), CuI (20 mg, 0.1 mmol), N,N-dimethylglycine.HCl (15 mg, 0.1 mmol), Cs₂CO₃ (128 mg, 0.4 mmol), dioxane (2 mL) and DMF (0.5 mL). The reaction was run in the microwave at 160° C. for 30 min. The solvent was evaporated and EtOAc and water were added. The water layer was extracted once with EtOAc and the combined organic layers were washed with brine. The organic layers were then dried over MgSO₄ and concentrated. The crude product was purified by flash chromatography eluting with 70:30 hexanes/EtOAc to give 110 mg (100%) of the title compound.

LC/MS: m/z 542 (M+H) Rt 2.88 min

### Intermediate 21: 3-[1-(ethylsulfonyl)-4-piperidinyl]-5-[(4-methoxyphenyl)oxy]-1H-indole-7-carbonitrile

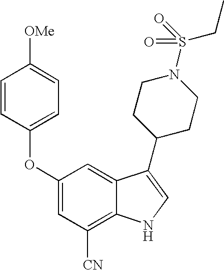

TBAF (0.77 mL, 0.77 mmol) was added to the solution of 3-[1-(ethylsulfonyl)-4-piperidinyl]-5-[(4-methoxyphenyl)oxy]-1-({[2-(trimethylsilyl)ethyl]oxy}methyl)-1H-indole-7-carbonitrile (110 mg, 0.19 mmol) in THF (10 mL). The reaction was maintained overnight at 65° C., followed by addition of EtOAc and water. It was then extracted once using EtOAc and dried over (MgSO₄). The organic layers were dried and concentrated. The crude product was purified by flash chromatography eluting with 2:1 hexanes/EtOAc to give 42 mg (50%) of the title compound.

LC/MS: m/z 440 (M+H) Rt 2.27 min

### Intermediate 22: 5-{[3-(diethylamino)phenyl]oxy}-3-[1-(ethylsulfonyl)-4-piperidinyl]-1-({[2-(trimethylsilyl)ethyl]oxy}methyl)-1H-indole-7-carbonitrile

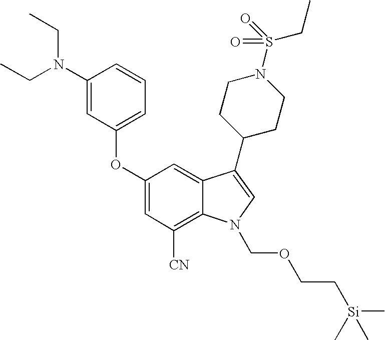

To a microwave vessel was added 5-bromo-3-[1-(ethylsulfonyl)-4-piperidinyl]-1-({[2-(trimethylsilyl)ethyl]oxy}methyl)-1H-indole-7-carbonitrile (100 mg, 0.2 mmol), 3-diethylamino phenol (63 mg, 0.4 mmol), CuI (20 mg, 0.1 mmol), N,N-dimethylglycine.HCl (16 mg, 0.1 mmol), Cs₂CO₃ (125 mg, 0.4 mmol), dioxane (2 mL) and DMF (0.5 mL). The reaction was run in the microwave at 160° C. for 30 min. The solvent was evaporated and EtOAc and water were added. The water layer was extracted once with EtOAc and the combined organic layers were washed with brine. The organic layers were then dried over MgSO₄ and concentrated. The crude product was purified by flash chromatography eluting with 3:1 hexanes/EtOAc to give 45 mg (43%) of the title compound.

LC/MS: m/z 598 (M+H) Rt 2.88 min

### Intermediate 23: 5-{[3-(diethylamino)phenyl]oxy}-3-[1-(ethylsulfonyl)-4-piperidinyl]-1H-indole-7-carbonitrile

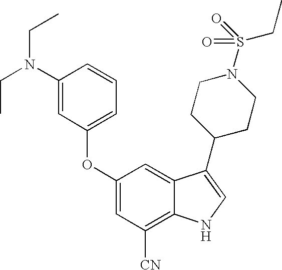

TBAF (0.29 mL, 0.29 mmol) was added to the solution of 5-{[3-(diethylamino)phenyl]oxy}-3-[1-(ethylsulfonyl)-4-piperidinyl]-1-({[2-(trimethylsilyl)ethyl]oxy}methyl)-1H-indole-7-carbonitrile (45 mg, 0.07 mmol) in THF (5 mL). The reaction was maintained overnight at 65° C., followed by addition of EtOAc and water. The layers were separated and the aqueous layer was extracted with EtOAc. The combined organic layers were dried (MgSO₄) and concentrated. Flash column (hexanes/EtOAc 2/1) to give 30 mg product (85%).

LC/MS: m/z 481 (M+H) Rt 1.78 min

### Intermediate 24: 3-[1-(ethylsulfonyl)-4-piperidinyl]-5-[(4-fluorophenyl)oxy]-1-({[2-(trimethylsilyl)ethyl]oxy}methyl)-1H-indole-7-carbonitrile

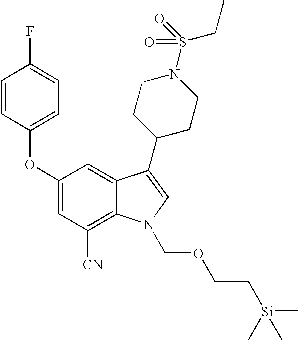

4-Fluorophenol (23 mg, 0.2 mmol), CuI (10 mg, 0.05 mmol), N,N-dimethylglycine.HCl (8 mg, 0.05 mmol), and Cs₂CO₃ (64 mg, 0.2 mmol) were added to a solution of 5-bromo-3-[1-(ethylsulfonyl)-4-piperidinyl]-1-({[2-(trimethylsilyl)ethyl]oxy}methyl)-1H-indole-7-carbonitrile in a 4:1 solution of dioxane/H₂O (2.5 mL) in a microwave vessel. The reaction was heated in a microwave at 160° C. for 30 min. The solvent was evaporated and EtOAc and water were added. The layers were separated and the aqueous layer was extracted once with EtOAc. The combined organic layers were washed with brine, dried (MgSO₄), and concentrated to give 50 mg (89%) of the title compound, which is used in the next step without purification.

¹H NMR (400 MHz, DMSOd₆) δ 7.93 (d, 1H), 7.66 (2, 1H), 7.07 (s, 1H), 5.72 (s, 2H), 4.00 (d, 2H), 3.57 (t, 2H), 2.87-3.06 (m, 5H), 1.76-1.86 (m, 2H), 1.42 (t, 3H), 1.28 (t, 2H), 0.00 (s, 9H)

### Intermediate 25: 3-[1-(ethylsulfonyl)-4-piperidinyl]-5-[(4-fluorophenyl)oxy]-1H-indole-7-carbonitrile

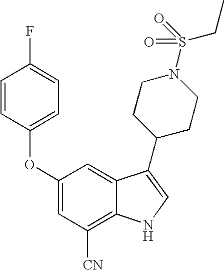

TBAF (0.27 mL, 0.27 mmol) was added to a solution of 3-[1-(ethylsulfonyl)-4-piperidinyl]-5-[(4-fluorophenyl)oxy]-1-({[2-(trimethyisilyl)ethyl]oxy}methyl)-1H-indole-7-carbonitrile (50 mg, 0.089 mmol) in THF (5 mL). The reaction was heated overnight at 50° C. EtOAc and water were added and the layers were separated. The aqueous layer was extracted with EtOAc, dried (MgSO₄), and concentrated to give 8 mg (16.3%) of the title compound, which is used in the next step without purification.

LC/MS: m/z 598 (M+H) Rt 2.88 min.

### Intermediate 26: 3-[1-(ethylsulfonyl)-4-piperidinyl]-5-[methyl(phenyl)amino]-1-({[2-(trimethylsilyl)ethyl]oxy}methyl)-1H-indole-7-carbonitrile

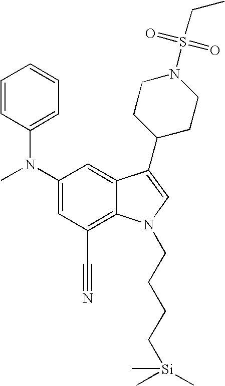

N-methylaniline (41 μL, 0.38 mmol), sodium tert-butoxide (27 mg, 0.28 mmol), tris(dibenzylideneacetone)dipalladium(0) (9.0 mg, 0.01 mmol), and 2-(di-t-butylphosphino)biphenyl (6.0 mg, 0.02 mmol) were added to a solution of 5-bromo-3-[1-(ethylsulfonyl)-4-piperidinyl]-1-({[2-(trimethylsilyl)ethyl]oxy}methyl)-1H-indole-7-carbonitrile (100 mg, 0.19 mmol) in tolulene (3 mL). The reaction was heated to 80° C. overnight and then cooled to room temperature. EtOAc, ether and water were then added followed by extraction with EtOAc. The combined organic layers were dried, concentrated and purified by flash chromatography eluting with (3:1) hexanes/EtOAc to give 60 mg (57%) of the title compound.

LC/MS=m/z 553 [M+H] Rt 2.93 min

### Intermediate 27: 3-[1-(ethylsulfonyl)-4-piperidinyl]-5-[methyl(phenyl)amino]-1H-indole-7-carbonitrile

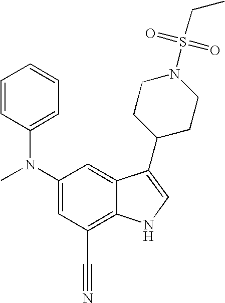

Tetra-n-butylammonium fluoride (0.4 mL, 0.4 mmol) was added to a solution of 3-[1-(ethylsulfonyl)-4-piperidinyl]-5-[methyl(phenyl)amino]-1-({[2-(trimethylsilyl)ethyl]oxy}methyl)-1H-indole-7-carbonitrile (60 mg, 0.1 mmol) in THF (10 mL), and the reaction was heated overnight. After cooling to room temperature, EtOAc and water were added to the mixture, followed by extraction with EtOAc. The combined organic layers were then dried, concentrated and purified using flash chromatography eluting with (2:1) hexane/EtOAc to give 30 mg (95%) of the title compound.

LC/MS=m/z 423 [M+H] Rt 2.40 min

### EXAMPLES

### Example 1

### 3-[1-(ethylsulfonyl)-4-piperidinyl]-5-hydroxy-1H-indole-7-carboxamide

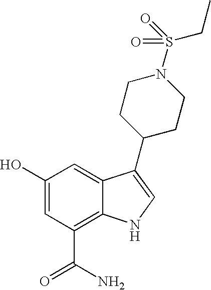

A 30% solution of hydrogen peroxide (0.012 mL, 0.4 mmol) and sodium hydroxide (3.96 mg, 0.099 mmol) were added to a solution of 3-[1-(ethylsulfonyl)-4-piperidinyl]-5-(4,4,5,5-tetramethyl-1,3,2-dioxaborolan-2-yl)-1H-indole-7-carboxamide (50 mg, 0.11 mmol) in DME (3 mL) at room temperature. The reaction mixture was stirred at room temperature for 1.5 hours. The solvent was evaporated and the residue was partitioned between ethyl acetate (25 mL) and water (25 mL). The aqueous layer was extracted with ethyl acetate (2×25 mL) and the combined organic phases were dried with Mg₂SO₄ and concentrated under reduce pressure. The crude product was purified by Gilson HPLC (reverse phase, eluting with CH₃CN/Water, 0.1% TFA, 10/90, v/v, over 15 min) to give 16.5 mg (43.2%) of the title compound.

LC-MS: m/z, 352.2 (M+H) Rt 1.49 min.

### Example 2

### 5-[(cyclopropylmethyl)oxy]-3-[1-(ethylsulfonyl)-4-piperidinyl]-1H-indole-7-carboxamide

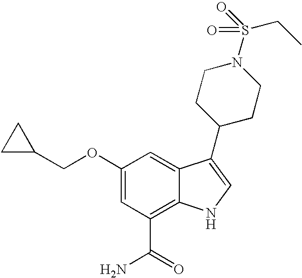

A solution of 1,1-dimethylethyl 7-[(bis{[(1,1-dimethylethyl)oxy]carbonyl}amino)carbonyl]-3-[1-(ethylsulfonyl)-4-piperidinyl]-5-hydroxy-1H-indole-1-carboxylate (65 mg, 0.1 mmol) in methylene chloride (1 mL) was treated with cyclopropylmethylbromide (28.9 μL, 0.3 mmol), water (1.0 mL), benzyl tri-n-butylammonium bromide (35.6 mg, 0.1 mmol), and NaOH (6 mg, 0.15 mmol). The reaction was stirred rapidly overnight at room temperature. The reaction mixture was washed with water and saturated. aqueous NaCl. The organic layer was dried (Na₂SO₄) and evaporated to give 97 mg of crude product. The crude product was taken up in methylene chloride (1 mL) and treated with TFA (1 mL). The solution stood for 1 h at room temperature and was stripped to dryness. The residue was taken up in EtOAc and washed with aqueous NaHCO₃. The organic layer was dried (Na₂SO₄) and evaporated to 62 mg of crude product, which was purified on a Chromatotron® silica gel plate (1000 u) eluting with 5% MeOH/CH₂Cl₂ to give the title compound 16.3 mg (40.2%) as a light, sandy brown solid.

LC/MS=m/z 406.6 [M+H]⁺ Rt 1.71 min.

### Example 3

### 3-[1-(ethylsulfonyl)-4-piperidinyl]-5-(pentyloxy)-1H-indole-7-carboxamide

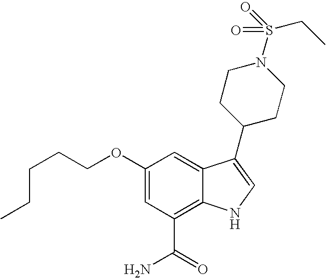

The title compound was prepared according to the general procedure of Example 2, substituting n-amyl bromide for cyclopropylmethyl bromide to give 19 mg (29%) of the title compound as a sand coloured solid.

LC/MS=m/z 422.4 [M+H]⁺ Rt 2.11 min.

### Example 4

### 3-[1-(ethylsulfonyl)-4-piperidinyl]-5-(octyloxy)-1H-indole-7-carboxamide

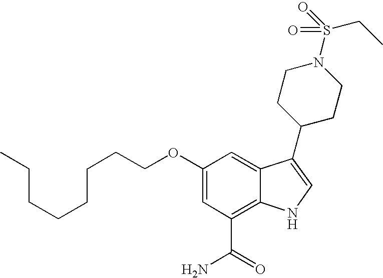

The title compound was prepared according to the general procedure of Example 2, substituting n-octyl bromide for cyclopropylmethyl bromide to give 18.1 mg (25%) of the title compound as a sand coloured solid.

LC/MS=m/z 464.4 [M+H]⁺ Rt 2.53 min.

### Example 5

### 3-[1-(ethylsulfonyl)-4-piperidinyl]-5-(heptyloxy)-1H-indole-7-carboxamide

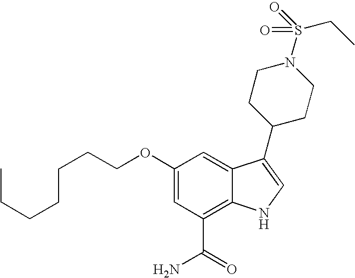

The title compound was prepared according to the general procedure of Example 2, substituting n-heptyl bromide for cyclopropylmethyl bromide to give 27.3 mg (39.4%) of the title compound as a sand coloured solid.

LC/MS=m/z 450.2 [M+H]⁺ Rt 2.41 min.

### Example 6

### 3-[1-(ethylsulfonyl)-4-piperidinyl]-5-[(2-phenylethyl)oxy]-1H-indole-7-carboxamide

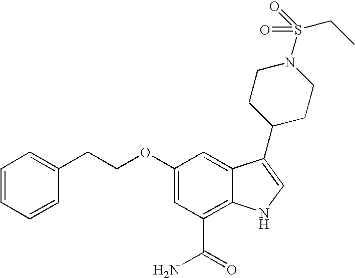

The title compound was prepared according to the general procedure of Example 2, substituting (2-bromoethyl)benzene for cyclopropylmethyl bromide to give 9.5 mg (13%) of the title compound as a sand coloured solid.

LC/MS=m/z 456.2 [M+H]⁺ Rt 2.06 min.

### Example 7

### 3-[1-(ethylsulfonyl)-4-piperidinyl]-5-[(3-phenylpropyl)oxy]-1H-indole-7-carboxamide

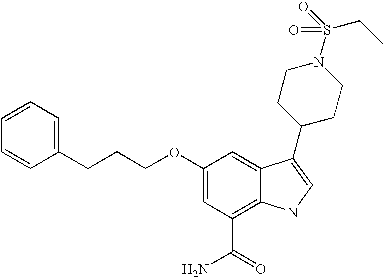

The title compound was prepared according to the general procedure of Example 2, substituting (3-bromopropyl)benzene for cyclopropylmethyl bromide to give 32.7 mg (45%) of the title compound as an ivory coloured solid.

LC/MS=m/z 470.4 [M+H]⁺Rt 2.18 min.

### Example 8

### 5-[(2-chloroethyl)oxy]-3-[1-(ethylsulfonyl)-4-piperidinyl]-1H-indole-7-carboxamide

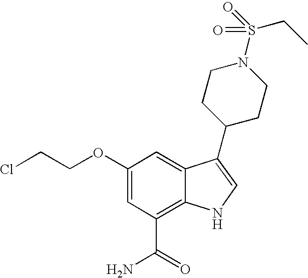

The title compound was prepared according to the general procedure of Example 2, substituting 2-chloroethylbromide for cyclopropylmethyl bromide to give 18.7 mg (29%) of the title compound as an ivory coloured solid.

LC/MS=m/z 414.2 [M+H]⁺ Rt 1.65 min.

### Example 9

### 3-[1-(ethylsulfonyl)-4-piperidinyl]-5-({4-[(phenylmethyl)oxy]butyl}oxy)-1H-indole-7-carboxamide

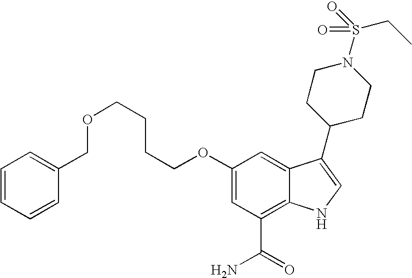

The title compound was prepared in the manner described in Example 2, substituting 4-bromobutyl-phenylmethylether for cyclopropylmethyl bromide to give 15.7 mg (20%) of the title compound as a creamy white solid.

LC/MS=m/z 514.6 [M+H]⁺ Rt 2.18 min.

### Example 10

### 3-[1-(ethylsulfonyl)-4-piperidinyl]-5-({2-[(phenyl methyl)oxy]ethyl}oxy)-1H-indole-7-carboxamide

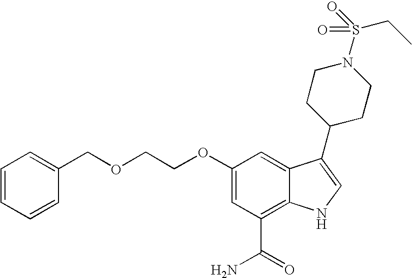

The title compound was prepared according to the general procedure of Example 2, substituting 2-bromoethyl-phenylmethylether for cyclopropylmethyl bromide to give 29.2 mg (39.1%) of the title compound as a light sand coloured solid.

LC/MS=m/z 486.2 [M+H]⁺ Rt 1.97 min.

### Example 11

### 5-[(3-cyanopropyl)oxy]-3-[1-(ethylsulfonyl)-4-piperidinyl]-1H-indole-7-carboxamide

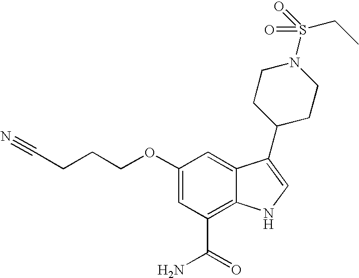

The title compound was prepared according to the general procedure of Example 2, substituting 4-bromobutanenitrile for cyclopropylmethyl bromide to give 30.3 mg (47%) of the title compound as a light coloured solid.

LC/MS=m/z 419.4 [M+H]⁺ Rt 1.46 min.

### Example 12

### 5-[(4-amino-4-oxobutyl)oxy]-3-[1-(ethylsulfonyl)-4-piperidinyl]-1H-indole-7-carboxamide

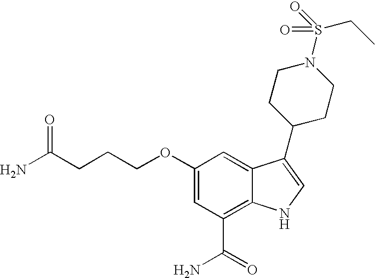

NaBO₃.4H₂O (43 mg, 0.28 mmol) was added to a solution of [5-[(3-cyanopropyl)oxy]-3-[1-(ethylsulfonyl)-4-piperidinyl]-1H-indole-7-carboxamide] (27 mg, 0.0645 mmol) in 1:1 EtOH/H₂O (2 mL). The reaction was heated in a microwave at 150° C. for 1 h. The reaction was stripped to dryness, taken up in EtOAc, and washed with H₂O. The aqueous phase was back-extracted with EtOAc, and the combined EtOAc layers were dried (Na₂SO₄) and evaporated. The residue was purified on a Chromatotron® silica gel plate (1000 u) eluting with 5% MeOH/CH₂Cl₂ to give a pure fraction of the title compound, which was triturated with CH₂Cl₂/MeOH, then evaporated to dryness to give 6.3 mg (22.3%) of the title compound as a white/ivory coloured solid.

LC/MS=m/z 437.4 [M+H]⁺ Rt 1.20 min.

### Example 13

### 3-[1-(ethylsulfonyl)-4-piperidinyl]-5-[(phenylmethyl)oxy]-1H-indole-7-carboxamide

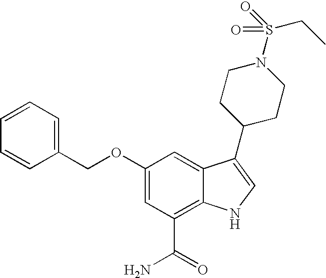

Trifluoroacetic acid (4 mL) was added to a solution of 1,1-dimethylethyl 7-[(bis{[(1,1-dimethylethyl)oxy]carbonyl}amino)carbonyl]-3-[1-(ethylsulfonyl)-4-piperidinyl]-5-[(phenylmethyl)oxy]-1H-indole-1-carboxylate (110 mg, 0.148 mmol) in CH₂Cl₂ (4 mL). The solution was stirred for 1 h and stripped to dryness. The residue was triturated with ethyl ether and dried to give the 35 mg of the crude product, which was further purified on a Chromatotron® silica gel plate (1000 u) eluting with 5% MeOH/CH₂Cl₂ to give a fraction of the title compound. Crystallization of the evaporated residue from a very small amount of MeOH—H₂O (75:25) gave 16 mg (24%) of the title compound as a pale grey solid.

LC/MS=m/z 442 [M+H]⁺ Rt 2.00 min.

### Example 14

### 3-[1-(ethylsulfonyl)-4-piperidinyl]-5-[(phenylmethyl)oxy]-1H-indole-7-carboxamide

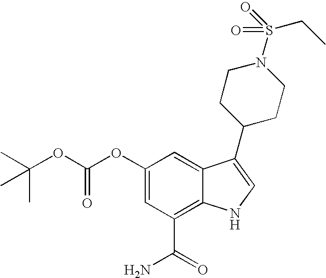

BOC anhydride (624 mg, 2.9 mmol) and dimethylaminopyridine (DMAP) (34 mg, 0.28 mmol) were added to a solution of 3-[1-(ethylsulfonyl)-4-piperidinyl]-5-hydroxy-1H-indole-7-carboxamide (289 mg, 0.822 mmol) in a 2:2:1 mixture of dry CH₂Cl₂/dry CH₃CN/dry THF (25 mL). The reaction was stirred overnight at room temperature. The reaction mixture was stripped to dryness and the residue was triturated with EtOAc to give 236 mg (64%) of the title compound as an ivory coloured crystalline solid.

LC/MS=m/z 352 (loss of BOC) [M+H]⁺ Rt 1.81 min.

¹H NMR (400 MHz, DMSO-d6) δ11.0 (s, 1H), 8.1 (bs, 1H), 7.61 (d, 1H), 7.54 (d, 1H), 7.42 (bs, 1H), 7.2 (d, 1H), 3.60-3.80 (m, 2H), 3.10 (q, 2H), 2.85-3.05 (m, 3H), 2.00-2.10 (d, 2H), 1.55-1.80 (m, 2H), 1.52 (s, 9H), 1.23-1.25 (t, 3H).

### Example 15

### 5-{[(3,4-difluorophenyl)methyl]oxy}-3-[1-(ethylsulfonyl)-4-piperidinyl]-1H-indole-7-carboxamide

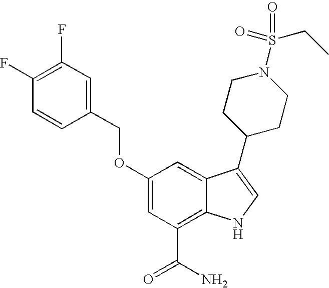

The title compound was prepared according to the general procedure of Example 2, substituting 4-(bromomethyl)-1,2-difluorobenzene for cyclopropylmethyl bromide to afford 17 mg (36%) of the title compound as a light coloured solid.

LC/MS=m/z 478 [M+H]⁺ Rt 2.11 min.

### Example 16

### 5-{[(3-chlorophenyl)methyl]oxy}-3-[1-(ethylsulfonyl)-4-piperidinyl]-1H-indole-7-carboxamide

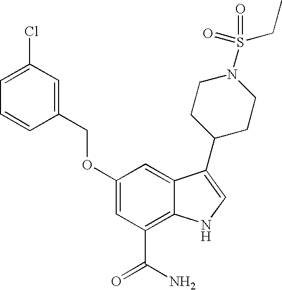

The title compound was prepared according to the general procedure of Example 2, substituting 1-(bromomethyl)-3-chlorobenzene for cyclopropylmethyl bromide to afford 20 mg (44%) of the title compound as a light coloured solid.

LC/MS=m/z 476 [M+H]⁺ Rt 2.20 min.

### Example 17

### methyl 4-[({7-(aminocarbonyl)-3-[1-(ethylsulfonyl)-4-piperidinyl]-1H-indol-5-yl}oxy)methyl]benzoate

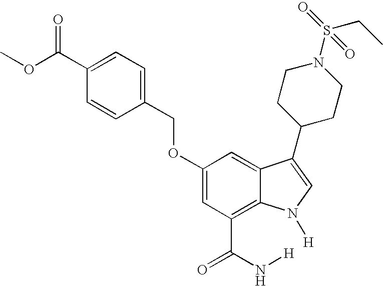

The title compound was prepared according to the general procedure of Example 2, substituting methyl 4-(bromomethyl)benzoate for cyclopropylmethyl bromide to afford 8.0 mg (16%) of the title compound as a light colored solid.

LC/MS=m/z 500 [M+H]⁺ Rt 2.06 min.

### Example 18

### 3-[1-(ethylsulfonyl)-4-piperidinyl]-5-{[(4-fluorophenyl)methyl]oxy}-1H-indole-7-carboxamide

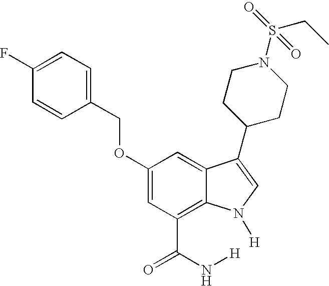

The title compound was prepared according to the general procedure of Example 2, substituting 1-(bromomethyl)-4-fluorobenzene for cyclopropylmethyl bromide to afford 10 mg (22%) of the title compound as a light colored solid.

LC/MS=m/z 460 [M+H]⁺ Rt 2.09 min.

### Example 19

### 5-{[(3-cyanophenyl)methyl]oxy}-3-[1-(ethylsulfonyl)-4-piperidinyl]-1H-indole-7-carboxamide

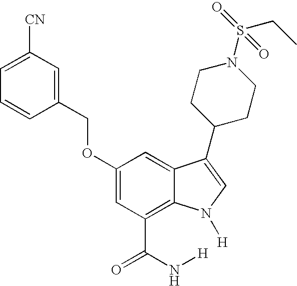

The title compound was prepared according to the general procedure of Example 2, substituting 3-(bromomethyl)benzonitrile for cyclopropylmethyl bromide to afford 15 mg (32%) of the title compound as a light colored solid.

LC/MS=m/z 467 [M+H]⁺ Rt 2.00 min.

### Example 20

### 3-[1-(ethylsulfonyl)-4-piperidinyl]-5-[({2-[(phenylsulfonyl)methyl]phenyl}methyl)oxy]-1H-indole-7-carboxamide

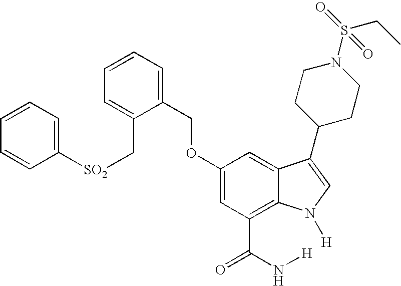

The title compound was prepared according to the general procedure of Example 2, substituting 1-(bromomethyl)-2-[(phenylsulfonyl)methyl]benzene for cyclopropylmethyl bromide to afford 20 mg (34%) of the title compound as a light colored solid.

LC/MS=m/z 596 [M+H]⁺ Rt 2.11 min.

### Example 21

### 5-{[(2-cyanophenyl)methyl]oxy}-3-[1-(ethylsulfonyl)-4-piperidinyl]-1H-indole-7-carboxamide

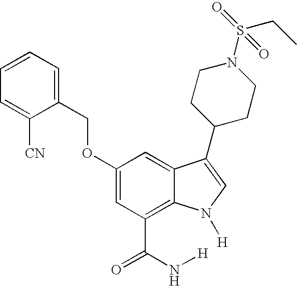

The title compound was prepared according to the general procedure of Example 2, substituting 2-(bromomethyl)benzonitrile for cyclopropylmethyl bromide to afford 25 mg (54%) of the title compound as a light colored solid.

LC/MS=m/z 467 [M+H]⁺ Rt 1.96 min.

### Example 22

### 3-[1-(ethylsulfonyl)-4-piperidinyl]-5-[(2-naphthalenylmethyl)oxy]-1H-indole-7-carboxamide

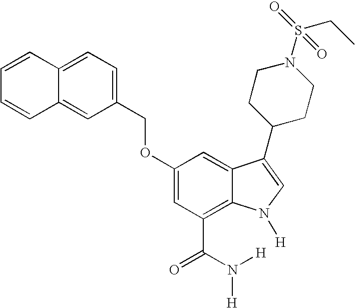

The title compound was prepared according to the general procedure of Example 2, substituting 2-(bromomethyl)naphthalene for cyclopropylmethyl bromide to afford 5 mg (10%) of the title compound as a light colored solid.

LC/MS=m/z 492 [M+H]⁺ Rt 2.05 min.

### Example 23

### 3-[1-(ethylsulfonyl)-4-piperidinyl]-5-[({3-[(trifluoromethyl)oxy]phenyl}methyl)oxy]-1H-indole-7-carboxamide

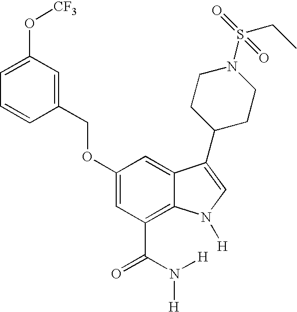

The title compound was prepared according to the general procedure of Example 2, substituting 3-(bromomethyl)phenyl trifluoromethyl ether for cyclopropylmethyl bromide to afford 15 mg (29%) of the title compound as a light colored solid.

LC/MS=m/z 526 [M+H]⁺ Rt 2.35 min.

### Example 24

### 3-[1-(ethylsulfonyl)-4-piperidinyl]-5-({[2-fluoro-4-(trifluoromethyl)phenyl]methyl}oxy)-1H-indole-7-carboxamide

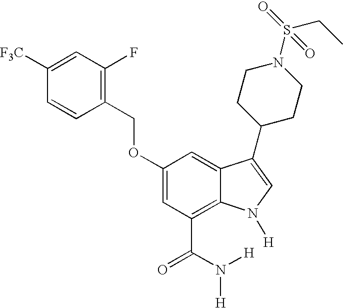

The title compound was prepared according to the general procedure of Example 2, substituting 1-(bromomethyl)-2-fluoro-4-(trifluoromethyl)benzene for cyclopropylmethyl bromide to afford 15 mg (28%) of the title compound as a light colored solid.

LC/MS=m/z 528 [M+H]⁺ Rt 2.41 min.

### Example 25

### 5-{[(3,5-difluorophenyl)methyl]oxy}-3-[1-(ethylsulfonyl)-4-piperidinyl]-1H-indole-7-carboxamide

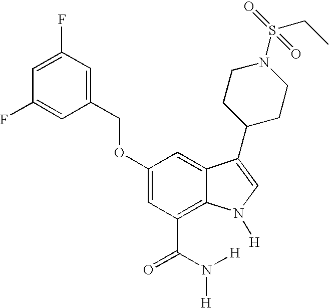

The title compound was prepared according to the general procedure of Example 2, substituting 1-(bromomethyl)-3,5-difluorobenzene for cyclopropylmethyl bromide to afford 17 mg (36%) of the title compound as a light colored solid.

LC/MS=m/z 478 [M+H]⁺ Rt 2.15 min.

### Example 26

### 5-[({3-[(difluoromethyl)oxy]phenyl}methyl)oxy]-3-[1-(ethylsulfonyl)-4-piperidinyl]-1H-indole-7-carboxamide

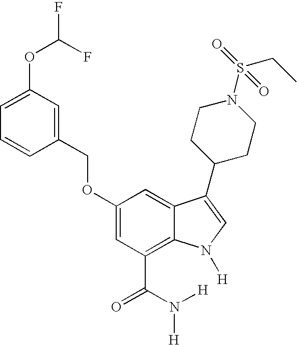

The title compound was prepared according to the general procedure of Example 2, substituting 3-(bromomethyl)phenyl difluoromethyl ether for cyclopropylmethyl bromide to afford 14 mg (28%) of the title compound as a light colored solid.

LC/MS=m/z 508 [M+H]⁺ Rt 2.26 min.

### Example 27

### 5-{[(3,4-dichlorophenyl)methyl]oxy}-3-[1-(ethylsulfonyl)-4-piperidinyl]-1H-indole-7-carboxamide

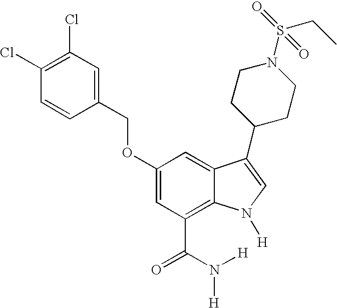

The title compound was prepared according to the general procedure of Example 2, substituting 4-(bromomethyl)-1,2-dichlorobenzene for cyclopropylmethyl bromide to afford 10 mg (21%) of the title compound as a light colored solid.

LC/MS=m/z 510 [M+H]⁺ Rt 2.35 min.

### Example 28

### 5-{[(4-chlorophenyl)methyl]oxy}-3-[1-(ethylsulfonyl)-4-piperidinyl]-1H-indole-7-carboxamide

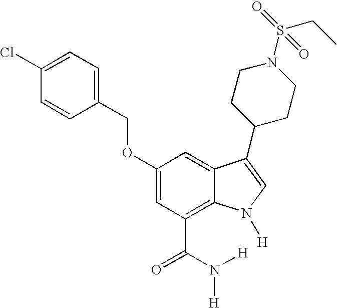

The title compound was prepared according to the general procedure of Example 2, substituting 4-(bromomethyl)-chlorobenzene for cyclopropylmethyl bromide to afford 17 mg (37%) of the title compound as a light colored solid.

LC/MS=m/z 476 [M+H]⁺ Rt 2.30 min.

### Example 29

### 3-[1-(ethylsulfonyl)-4-piperidinyl]-5-[(4-methyl phenyl)oxy]-1H-indole-7-carboxamide

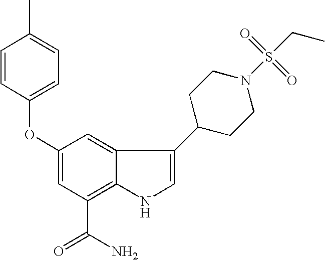

To 3-[1-(ethylsulfonyl)-4-piperidinyl]-5-[(4-methylphenyl)oxy]-1H-indole-7-carbonitrile (38 mg, 0.09 mmol) was added sodium perborate tetrahydrate (55 mg, 0.36 mmol) and a solution of 1:2 ethanol/water (3 mL). The reaction was heated in a microwave at 150° C. for 1.5 h. The solvent was evaporated, and the residue was taken up in EtOAc and water. The layers were separated, and the aqueous layer was extracted with EtOAc. The combined organic layers were dried, concentrated, and purified by flash chromatography eluting with (1:3) hexanes/EtOAc to give 5 mg (13%) of the title compound.

LC/MS=m/z 442 [M+H] Rt 2.21 min

### Example 30

### 3-[1-(ethylsulfonyl)-4-piperidinyl]-5-{[4-(methyloxy)phenyl]oxy}-1H-indole-7-carboxamide

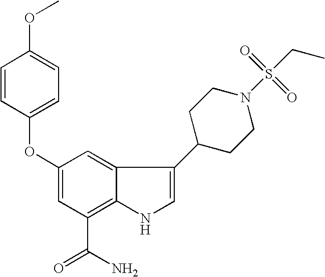

To 3-[1-(ethylsulfonyl)-4-piperidinyl]-5-{[4-(methyloxy)phenyl]oxy}-1H-indole-7-carbonitrile (42 mg, 0.096 mmol) was added sodium perborate tetrahydrate (58 mg, 0.38 mmol) and a solution of 1:2 ethanol/water (3 mL). The reaction was heated in a microwave at 150° C. for 30 min. The solvent was evaporated, and the residue was taken up in EtOAc and water. The layers were separated, and the aqueous layer was extracted with EtOAc. The combined organic layers were dried, concentrated, and purified by flash chromatography eluting with (1:2) hexanes/EtOAc to give 10 mg (23%) of the title compound.

LC/MS=m/z 458 [M+H] Rt 2.00 min

### Example 31

### 5-{[3-(diethylamino)phenyl]oxy}-3-[1-(ethylsulfonyl)-4-piperidinyl]-1H-indole-7-carboxamide

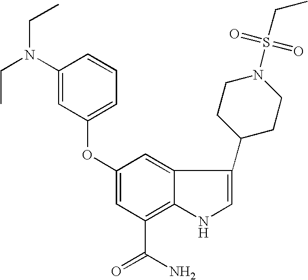

To 5-{[3-(diethylamino)phenyl]oxy}-3-[1-(ethylsulfonyl)-4-piperidinyl]-1H-indole-7-carbonitrile was added perborate tetrahydrate (38 mg, 0.25 mmol) and a solution of 1:2 EtOAc/water (3 mL). The reaction was heated in a microwave at 150° C. for 30 min. The solvent was evaporated, and the residue was taken up in EtOAc and water. The layers were separated, and the aqueous layer was extracted with EtOAc. The combined organic layers were concentrated and purified by flash chromatography eluting with (1:3) hexanes/EtOAc to give 10 mg (8%) of the title compound.

LC/MS=m/z 499 [M+H] Rt 1.68 min

### Example 32

### 3-[1-(ethylsulfonyl)-4-piperidinyl]-5-[(4-fluorophenyl)oxy]-1H-indole-7-carboxamide

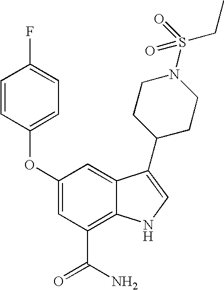

To 3-[1-(ethylsulfonyl)-4-piperidinyl]-5-[(4-fluorophenyl)oxy]-1H-indole-7-carbonitrile (13 mg, 0.03 mmol) was added sodium perborate tetrahydrate (19 mg, 0.12 mmol) and (1:2) ethanol and water (3 mL). The resulting mixture was reacted in a microwave at 150° C. for 30 min. The solvent was evaporated, and the residue was taken up in EtOAc and water. The layers were separated, and the aqueous layer was extracted with EtOAc. The combined organic layers were dried and concentrated to give 7 mg (52%) of the title compound as a yellow solid. The title compound was of sufficient purity to carry on to the next step without further purification.

LC/MS: m/z 446 (M+H) Rt 2.08 min

### Example 33

### 3-[1-(ethylsulfonyl)-4-piperidinyl]-5-[(2-methylpropyl)amino]-1H-indole-7-carboxamide

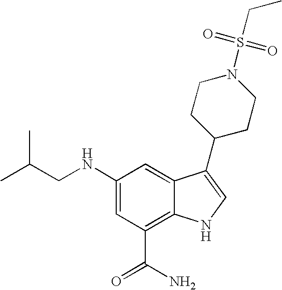

To 3-[1-(ethylsulfonyl)-4-piperidinyl]-5-[(2-methylpropyl)amino]-1-({[2-(trimethylsilyl)ethyl]oxy}methyl)-1H-indole-7-carbonitrile (32 mg, 0.06 mmol) was added 98% sulfuric acid (0.1 mL) plus one drop of water at room temperature. The reaction was heated at 80° C. for 30 min. Saturated aqueous sodium bicarbonate and EtOAc were added, and the reaction was adjusted to basic pH with 2 M sodium hydroxide. EtOAc was added, the layers were separated, and the organic layers was dried and concentrated. The crude product was purified by flash chromatography eluting with (1:2) hexane/EtOAc to yield 10 mg (41%) of the title compound.

LC/MS=m/z 407 [M+H] Rt 1.41 min

### Example 34

### 3-[1-(ethylsulfonyl)-4-piperidinyl]-5-[methyl(phenyl)amino]-1H-indole-7-carboxamide

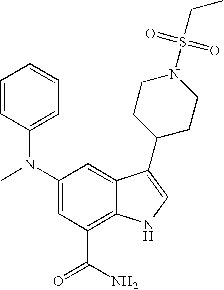

Sodium perborate tetrahydrate (44 mg, 0.28 mmol) was added to a solution of 3-[1-(ethylsulfonyl)-4-piperidinyl]-5-[methyl(phenyl)amino]-1H-indole-7-carbonitrile (30 mg, 0.071 mmol) in 1:2 ethanol/water (3 mL). The reaction was heated in a microwave at 150° C. for 1 h. The solvent was evaporated and EtOAc and water were added. The layers were separated, and the aqueous layer was extracted with EtOAc, dried and concentrated. The crude product was purified by flash chromatography to afford 15 mg (48%) of the title compound.

LC/MS=m/z 441 [M+H] Rt 2.11 min

### Example 35

### 3-[1-(ethylsulfonyl)piperidin-4-yl]-5-(phenylthio)-1H-indole-7-carboxamide

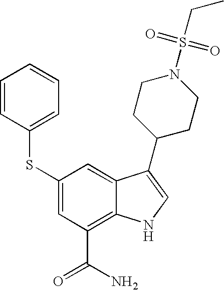

Ethylene glycol (10 μL, 0.29 mmol), potassium carbonate (41 mg, 0.29 mmol), copper iodide (3 mg, 0.015 mmol), and thiophenol (30 mL, 0.29 mmol were added to a solution of 5-bromo-3-[1-(ethanesulfonyl)-4-piperidinyl]-1H-indole-7-carboxamide (61 mg, 0.15 mmol) in isopropanol (2 mL). The reaction mixture was heated in a microwave at 180° C. for 3.5 h. The solvent was evaporated, and the residue was taken up in EtOAc and H₂O. The layers were separated, and the aqueous layer was extracted with EtOAc. The combined organic layers were dried, concentrated, and the crude product was purified by flash chromatography eluting with hexane/EtOAc (1:2). The partially purified product from the chromatography was re-purified on Gilson preparatory HPLC to give 9.0 mg (7%) of the title compound.

LC/MS=m/z 444 [M+H] Rt 2.00 min.

### Example 36

### 5-[(4-chlorophenyl)thio]-3-[1-(ethylsulfonyl)-4-piperidinyl]-1H-indole-7-carboxamide

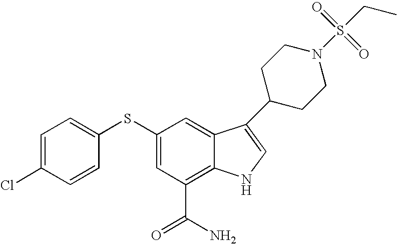

To a solution of 5-bromo-3-[1-(ethanesulfonyl)-4-piperidinyl]-1H-indole-7-carboxamide (66 mg, 0.15 mmol) in isopropanol (2 mL) was added chlorothiophenol (46 mg, 0.32 mmol), ethylene glycol (18 μL, 0.32 mmol), copper iodide (4 mg, 0.016 mmol) and potassium carbonate (45 mg, 0.032 mmol). The reaction was heated in a microwave at 180° C. for 1 h and then 190° C. for an additional 3 h. The solvent was evaporated, and EtOAc and H₂O were added. The layers were separated, and the aqueous layer was extracted with EtOAc. The combined organic layers were dried, concentrated, and purified by Gilson Prepatory HPLC to give 15 mg (10%) of the title compound.

LC/MS=m/z 478 [M+H] Rt 2.29 min

### Example 37

### 5-[(2-chlorophenyl)thio]-3-[1-(ethylsulfonyl)-4-piperidinyl]-1H-indole-7-carboxamide

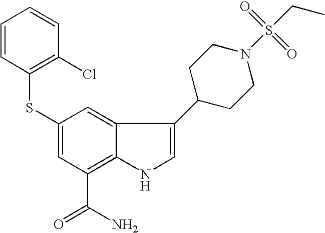

To a solution of 5-bromo-3-[1-(ethanesulfonyl)-4-piperidinyl]-1H-indole-7-carboxamide (66 mg, 0.159 mmol) in isopropanol (2 mL) was added chlorothiophenol (46 mg, 0.30 mmol), ethylene glycol (18 μL, 0.32 mmol), copper iodide (4 mg, 0.016 mmol) and potassium carbonate (45 mg, 0.032 mmol). The reaction was heated in a microwave at 180° C. for 1 h. The solvent was evaporated, and EtOAc and H₂O were added. The layers were separated, and the aqueous layer was extracted with EtOAc. The combined organic layers were dried, concentrated, and purified by Gilson Prepatory HPLC to give 15 mg (26%) of the title compound.

LC/MS=m/z 478 [M+H] Rt 2.27 min

### Example 38

### 3-[1-(ethylsulfonyl)-4-piperidinyl]-5-[(4-methylphenyl)thio]-1H-indole-7-carboxamide

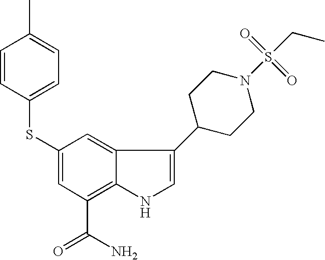

To a solution of 5-bromo-3-[1-(ethylsulfonyl)-4-piperidinyl]-1H-indole-7-carboxamide (160 mg, 0.36 mmol) in isopropanol (2 mL) was added p-thiocresol (90 mg, 0.72 mmol), ethylene glycol (40 μL, 0.726 mmol), copper iodide (7 mg, 0.037 mmol), and potassium carbonate (100 mg, 0.72 mmol). The mixture was heated in a CEM microwave at 160° C. for 1 h. The solvent was evaporated, and EtOAc and H₂O were added. The layers were separated, and the aqueous layer was extracted with EtOAc. The combined organic layers were concentrated and purified by flash chromatography eluting with (1:3) hexanes/EtOAc to give 10 mg (6%) of the title compound.

LC/MS=m/z 458 [M+H] Rt 2.22 min

### Example 39

### 3-[1-(ethylsulfonyl)-4-piperidinyl]-5-[(4-fluorophenyl)thio]-1H-indole-7-carboxamide

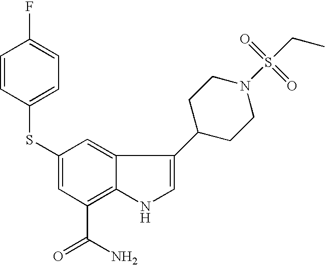

To a solution of 5-bromo-3-[1-(ethylsulfonyl)-4-piperidinyl]-1H-indole-7-carboxamide (150 mg, 0.36 mmol) in isopropanol (2 mL) was added 4-fluorothiophenol (78 mL, 0.72 mmol), ethylene glycol (40 μL, 0.726 mmol), copper iodide (7 mg, 0.037 mmol), and potassium carbonate (100 mg, 0.72 mmol). The mixture was heated in a microwave at 180° C. for 8 h. The solvent was evaporated, and EtOAc and H₂O were added. The layers were separated, and the aqueous layer was extracted with EtOAc. The combined organic layers were concentrated and purified by Gilson Prepatory HPLC to give 24 mg (14%) of the title compound.

LC/MS=m/z 462 [M+H] Rt 2.20 min

### Example 40

### 3-[1-(ethylsulfonyl)-4-piperidinyl]-5-[(3-fluorophenyl)thio]-1H-indole-7-carboxamide

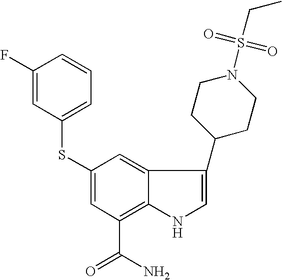

To a solution of 5-bromo-3-[1-(ethylsulfonyl)-4-piperidinyl]-1H-indole-7-carboxamide (150 mg, 0.36 mmol) in isopropanol (2 mL) was added 3-fluorothiophenol (70 mL, 0.72 mmol), ethylene glycol (40 μL, 0.726 mmol), copper iodide (7 mg, 0.037 mmol), and potassium carbonate (100 mg, 0.72 mmol). The reaction was heated in a microwave at 180° C. for 8 h. The solvent was evaporated, and EtOAc and H₂O were added. The layers were separated, and the aqueous layer was extracted with EtOAc. The combined organic layers were concentrated and purified by Gilson Prepatory HPLC to give 13 mg (8%) of the title compound.

LC/MS=m/z 462 [M+H] Rt 2.23 min

### Example 41

### 3-[1-(ethylsulfonyl)-4-piperidinyl]-5-[(3-fluorophenyl)thio]-1H-indole-7-carboxamide

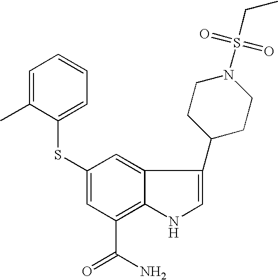

To a solution of 5-bromo-3-[1-(ethylsulfonyl)-4-piperidinyl]-1H-indole-7-carboxamide (150 mg, 0.36 mmol) in isopropanol (2 mL) was added o-thiocresol (86 mL, 0.72 mmol), ethylene glycol (40 μL, 0.726 mmol), copper iodide (7 mg, 0.037 mmol), and potassium carbonate (100 mg, 0.72 mmol). The reaction was heated in a microwave at 180° C. for 8 h. The solvent was evaporated, and EtOAc and H₂O were added. The layers were separated, and the aqueous layer was extracted with EtOAc. The combined organic layers were concentrated and purified by Gilson Prepatory HPLC to give 15 mg (9%) of the title compound.

LC/MS=m/z 458 [M+H] Rt 2.24 min

### Example 42

### 3-[1-(ethylsulfonyl)-4-piperidinyl]-5-[(2-fluorophenyl)thio]-1H-indole-7-carboxamide

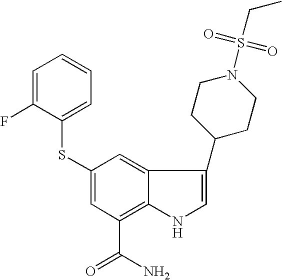

To a solution of 5-bromo-3-[1-(ethylsulfonyl)-4-piperidinyl]-1H-indole-7-carboxamide (150 mg, 0.36 mmol) in isopropanol (2 mL) was added 2-fluorothiophenol (78 mL, 0.72 mmol), ethylene glycol (40 μL, 0.726 mmol), copper iodide (7 mg, 0.037 mmol), and potassium carbonate (100 mg, 0.72 mmol). The reaction was heated in a microwave at 180° C. for 8 h. The solvent was evaporated, and EtOAc and H₂O were added. The layers were separated, and the aqueous layer was extracted with EtOAc. The combined organic layers were concentrated and purified by Gilson Prepatory HPLC to give 18 mg (11%) of the title compound.

LC/MS=m/z 462 [M+H] Rt 2.19 min

### Example 43

### 3-[1-(ethylsulfonyl)-4-piperidinyl]-5-[(3-methylphenyl)thio]-1H-indole-7-carboxamide

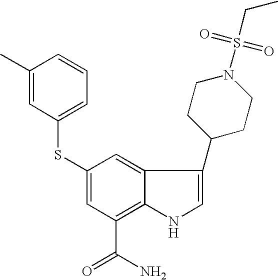

To a solution of 5-bromo-3-[1-(ethylsulfonyl)-4-piperidinyl]-1H-indole-7-carboxamide (150 mg, 0.36 mmol) in isopropanol (2 mL) was added m-thiocresol (86 mL, 0.72 mmol), ethylene glycol (40 μL, 0.726 mmol), copper iodide (7 mg, 0.037 mmol), potassium carbonate (100 mg, 0.72 mmol). The reaction was heated in a microwave at 180° C. for 8 h. The solvent was evaporated, and EtOAc and water were added to the residue. The layers were separated, and the organic layer was concentrated and purified by Gilson Prepatory HPLC to give 16 mg (3%) of the title compound.

LC/MS=m/z 458 [M+H] Rt 2.23 min

### Example 44

### 5-{[2-(diethylamino)ethyl]thio}-3-[1-(ethylsulfonyl)-4-piperidinyl]-1H-indole-7-carboxamide trifluoroacetate

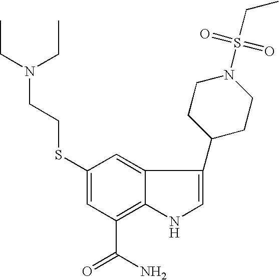

To a solution of 5-bromo-3-[1-(ethylsulfonyl)-4-piperidinyl]-1H-indole-7-carboxamide (70 mg, 0.169 mmol) in isopropanol (2 mL) was added 2-(diethylamino)-ethanethiol-hydrochloride (52 mg, 0.32 mmol), ethylene glycol (18 μL, 0.32 mmol), copper iodide (4 mg, 0.016 mmol), potassium carbonate (67 mg, 0.48 mmol). The reaction was heated in a microwave at 160° C. for 2 h. The solvent was evaporated and EtOAc and water were added to the residue. The layers were separated, and the organic layer was dried, concentrated and purified by Gilson Prepatory HPLC to give 5 mg (6%) of the title compound.

LC/MS=m/z 467 [M+H] Rt 1.60 min

### Example 45

### 5-[(2,4-dichlorophenyl)thio]-3-[1-(ethylsulfonyl)-4-piperidinyl]-1H-indole-7-carboxamide

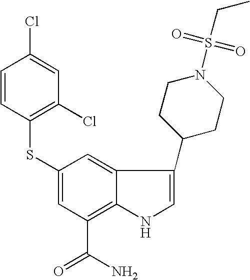

To 5-bromo-3-[1-(ethylsulfonyl)-4-piperidinyl]-1H-indole-7-carboxamide (100 mg, 0.24 mmol) in isopropanol (2 mL) was added 2,4-dichlorothiophenol (86 mg, 0.48 mmol), ethylene glycol (27 mg, 0.48 mmol), copper iodide (5 mg, 0.024 mmol), and potassium carbonate (67 mg, 0.48 mmol). The reaction was heated in a microwave at 160° C. for 3 h. The solvent was evaporated and EtOAc and water were added to the residue. The layers were separated, and the organic layer was concentrated and purified by Gilson Prepatory HPLC to give 22 mg (4%) of the title compound.

LC/MS=m/z 512 [M+H] Rt 2.40 min

### Example 46

### 3-∂1-(ethylsulfonyl)-4-piperidinyl-5-[(2-methylpropyl)thio]-1H-indole-7-carboxamide

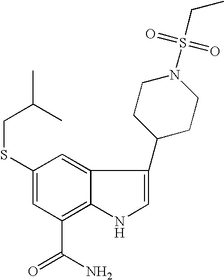

To 5-bromo-3-[1-(ethylsulfonyl)-4-piperidinyl]-1H-indole-7-carboxamide (150 mg, 0.36 mmol) in isopropanol (3 mL) was added 2-methyl-1-propanethiol (78 mL, 0.72 mmol), ethylene glycol (40 mL, 0.72 mmol), copper iodide (7 mg, 0.18 mmol), and potassium carbonate (100 mg, 0.72 mmol). The reaction was heated in a microwave at 140° C. for 170 min. The resultant mixture was purified by Gilson Prepatory HPLC to give 13 mg (9%) of the title compound.

LC/MS=m/z 424 [M+H] Rt 2.18 min

### Example 47

### 5-{[4-(acetylamino)phenyl]thio}-3-[1-(ethylsulfonyl)-4-piperidinyl]-1H-indole-7-carboxamide

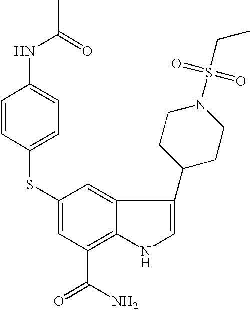

To 5-bromo-3-[1-(ethylsulfonyl)-4-piperidinyl]-1H-indole-7-carboxamide (100 mg, 0.24 mmol) in isopropanol (2 mL) was added 4-acetamidothiophenol (81 mg, 0.48 mmol), ethylene glycol (27 mL, 0.48 mmol), copper iodide (5 mg, 0.024 mmol), and potassium carbonate (67 mg, 0.48 mmol). The solvent was evaporated and EtOAc and water were added to the residue. The layers were separated, and the organic layer was concentrated and purified by Gilson Prepatory HPLC to give 10 mg (41.7%) of the title compound.

LC/MS=m/z 501 [M+H] Rt 1.89 min

### Example 48

### 3-[1-(ethylsulfonyl)-4-piperidinyl]-5-[(4-methylphenyl)sulfonyl]-1H-indole-7-carboxamide

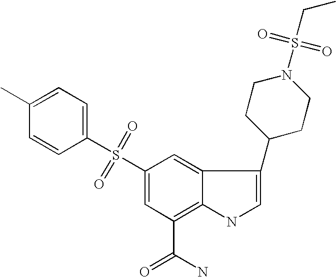

To 3-[1-(ethylsulfonyl)-4-piperidinyl]-5-[(4-methylphenyl)thio]-1H-indole-7-carboxamide (20 mg, 0.04 mmol) in MeOH (2 mL) was added a solution of oxone (81 mg, 0.13 mmol) in water (2 mL) at room temperature. The solvent was evaporated and aqueous sodium bicarbonate was added to the residue. The layers were separated, and the organic layer was dried, concentrated, and purified by Gilson Prepatory HPLC to give 6 mg (31%) of the title compound.

LC/MS=m/z 490 [M+H] Room temperature 1.99 min

### Example 49

### 3-[1-(ethylsulfonyl)-4-piperidinyl]-5-[(3-fluorophenyl)sulfonyl]-1H-indole-7-carboxamide

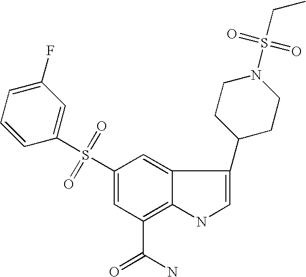

To a solution of 3-[1-(ethylsulfonyl)-4-piperidinyl]-5-[(3-fluorophenyl)sulfonyl]-1H-indole-7-carboxamide (24 mg, 0.052 mmol) in MeOH (2 mL) was added a solution of potassium hydrogen persulfate (96 mg, 0.156 mmol) in water (2 mL) at room temperature overnight. The solvent was evaporated and EtOAc, water and saturated aqueous sodium bicarbonate were added to the residue. The layers were separated, and the organic layer was dried, concentrated and purified by Gilson Prepatory HPLC to give 12 mg (47%) of the title compound.

LC/MS=m/z 494 [M+H] Rt 1.95

### Example 50

### 5-{[4-(acetylamino)phenyl]sulfonyl}-3-[1-(ethylsulfonyl)-4-piperidinyl]-1H-indole-7-carboxamide

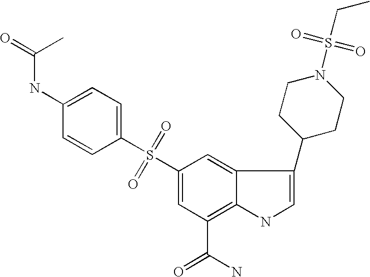

To a solution of 5-[(4-acetylphenyl)thio]-3-[1-(ethylsulfonyl)-4-piperidinyl]-1H-indole-7-carboxamide (33 mg, 0.066 mmol) in MeOH (5 mL) was added a solution of oxone (41 mg, 0.066 mmol) in H₂O (5 mL), and the reaction was stirred overnight. The solvent was evaporated and equal parts of EtOAc and saturated aqueous sodium bicarbonate were added to the residue. The layers were separated, and the organic layer was dried, concentrated and purified by Gilson Prepatory HPLC to give 5 mg (14%) of the title compound.

LC/MS=m/z 533 [M+H] Rt 1.71 min

### Biological Data

### IKK2 Assay

Recombinant human IKKβ (residues 1-737) was expressed in baculovirus as a C-terminal GST-tagged fusion protein, and its activity was assessed using a time-resolved fluorescence resonance energy transfer (TR-FRET) assay. Briefly, IKK2 (5 nM final) diluted in assay buffer (50 mM HEPES, 10 mM MgCl₂, 1 mM CHAPS pH 7.4 with 1 mM DTT and 0.01% w/v BSA) was added to wells containing various concentrations of compound or DMSO vehicle (3% final). The reaction was initiated by the addition of GST-IκBα substrate (25 nM final)/ATP (1 μM final), in a total volume of 30 μL. The reaction was incubated for 30 minutes at room temperature, then terminated by the addition of 15 μL of 50 mM EDTA. Detection reagent (15 μl) in buffer (100 mM HEPES pH 7.4, 150 mM NaCl and 0.1% w/v BSA) containing antiphosphoserine-licBa-32/36 monoclonal antibody 12C2 (Cell Signalling Technology, Beverly Mass., USA) labelled with W-1024 europium chelate (Wallac OY, Turku, Finland), and an APC-labelled anti-GST antibody (Prozyme, San Leandro, Calif., USA) was added and the reaction was further incubated for 60 minutes at room temperature. The degree of phosphorylation of GST-IκBα was measured using a Packard Discovery plate reader (Perkin-Elmer Life Sciences, Pangbourne, UK) as a ratio of specific 665 nm energy transfer signal to reference europium 620 nm signal.

### Results

The compounds of Examples 1-10 and 12-50 were tested for activity against IKK2 and were found to be inhibitors of IKK2. These examples had a pIC₅₀ of 5.0 or greater. Example 48 was tested and found to have a pIC₅₀ of 4.6.

### Monocyte Assay

Effect of IKK-β inhibition on human monocyte stimulated cytokine production was assessed as follows: Monocytes were isolated from heparinized whole blood by Ficoll gradient, followed by purification of CD14+ cells using MACS magnetic cell separation beads. Isolated monocytes were then adhered to 96-well culture plates at 1×10⁶ cells/mL in RPMI 1640 10% FBS (JRH Biosciences, Lenexa Kans.) for 2 h. to further enrich the monocyte population. The media was then removed, cells washed once with RPMI 1640, and 0.125 mL RPMI 1640 10% FBS was added to the wells. Test compounds are added to the wells 30 minutes prior to stimulation with a final vehicle concentration of 0.1% DMSO. Monocytes were activated by the addition of 200 ng/mL endotoxin (LPS; E. coli serotype 026:B6) (Sigma, St. Louis, Mo.) and incubated for 24 h at 37° C. Cell-free supernates were analyzed by ELISA for TNF-α using Pharmingen matched pair Abs. Viability of the cells was determined by 10% trypan blue exclusion.

What is claimed is:

1. A compound according to formula (I): 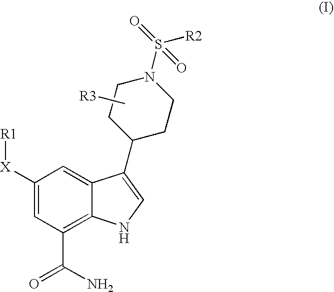
wherein:
X is O, S, S(O), S(O)₂, —N(Rf), or —OC(O)O;
R1 is H, optionally substituted C₁-C₈ alkyl, C₁-C₆ haloalkyl, optionally substituted heterocycloalkyl, optionally substituted —C₁-C₃ alkylene-heterocycloalkyl, optionally substituted phenyl, optionally substituted —C₁-C₃ alkylene-phenyl, optionally substituted naphthyl, optionally substituted —C₁-C₃ alkylene-naphthyl, optionally substituted heteroaryl, or optionally substituted —C₁-C₃ alkylene-heteroaryl,
where said C₁-C₈ alkyl is optionally substituted with one substituent selected from the group consisting of: cyano, —NRfRf, —C(O)NRfRf, C₃-C₆ cycloalkyl, and C₁-C₆ alkoxy optionally substituted with one phenyl group;
where said heterocycloalkyl and —C₁-C₃ alkylene-heterocycloalkyl are optionally substituted with one to three substituents each independently selected from the group consisting of: halo, hydroxyl, oxo, and C₁-C₆ alkyl;
where said phenyl, —C₁-C₃ alkylene-phenyl, heteroaryl, and —C₁-C₃ alkylene-heteroaryl, are each optionally substituted with one to three substituents each independently selected from the group consisting of: halo, —CN, —N(Rb)SO₂Re, —N(Rb)C(O)Ra, —C(O)NRaRb, —C(O)H, —SO₂Ri, —NRaRb, —SO₂NRaRb, —ORc, —N(Rb)C(O)NRaRb, —N(Rb)C(O)ORd, —C(O)ORa, C₁-C₆ alkyl, C₁-C₆ alkyl substituted with one to three substituents independently selected from the group consisting of: —NRaRb, C₃-C₆ cycloalkyl, phenyl, —ORc, heterocycloalkyl, and heterocycloalkyl substituted with OH, —C(O)NH₂, or one or two C₁-C₆ alkyl groups; C₁-C₆ haloalkyl, C₁-C₆ haloalkyl substituted with one to three substituents each independently selected from the group consisting of —NRaRb, C₃-C₆ cycloalkyl, phenyl, heterocycloalkyl, and heterocycloalkyl substituted with one or two C₁-C₆ alkyl groups; heterocycloalkyl and heterocycloalkyl substituted with one or two C₁-C₆ alkyl groups;
R2 is optionally substituted C₁-C₆ alkyl, optionally substituted aryl, optionally substituted C₃-C₆ cycloalkyl, optionally substituted heteroaryl, or optionally substituted heterocycloalkyl,
wherein said C₁-C₆ alkyl is optionally substituted with one to three substituents each independently selected from the group consisting of: halo, —OR1, —NRgRh, —NHC(O)Rg, and Rj; and where said aryl and heteroaryl are optionally substituted with one to three substituents each independently selected from the following: halo, —ORg, nitro, cyano, CF₃, C₁-C₆ alkyl, C(O)R9, COORg, —NRgRh, —NHC(O)Rg, —C(O)NRgRh, —S(O)₂Rg, —NHS(O)₂Rg, and —S(O)₂NRgRh; and where said C₃-C₆ cycloalkyl and heterocycloalkyl are optionally substituted by one to three substituents each independently selected from the group consisting of: —OH, oxo, C₁-C₆ alkyl, and C₁-C₆ haloalkyl;
R3 is one to three substituents each independently selected from the group consisting of: OH, oxo, C₁-C₆ alkyl, and C₁-C₆ haloalkyl;
each Ra is independently selected from the group consisting of: H, optionally substituted C₁-C₃ alkyl, optionally substituted phenyl, optionally substituted heteroaryl, optionally substituted C₃-C₇ cycloalkyl, and optionally substituted heterocycloalkyl, where said C₁-C₃alkyl is optionally substituted with one to three substituents each independently selected from the group consisting of: halo, ORc, C₁-C₆ haloalkyl, phenyl, and heteroaryl; and where said phenyl, heteroaryl, C₃-C₇ cycloalkyl, and heterocycloalkyl are optionally substituted with one to three substituents each independently selected from the group consisting of: halo, ORc, C₁-C₆ alkyl, and C₁-C₆ haloalkyl;
each Rb is independently selected from the group consisting of: H and optionally substituted C₁-C₃ alkyl, where said C₁-C₃ alkyl is optionally substituted with one to three ORc groups;
each Rc is independently selected from the group consisting of: H, optionally substituted C₁-C₆ alkyl, optionally substituted C₁-C₆ haloalkyl, optionally substituted C₃-C₇ cycloalkyl, optionally substituted heterocycloalkyl, optionally substituted aryl, and optionally substituted heteroaryl, where said C₁-C₆ alkyl and C₁-C₆ haloalkyl are optionally substituted with one to three substituents each independently selected from the group consisting of: C₃-C₆ cycloalkyl, phenyl, heterocycloalkyl, and heteroaryl; and where said aryl and heteroaryl are optionally substituted with one to three substituents each independently selected from the group consisting of: halo, C₁-C₃ alkyl, C₁-C₃ haloalkyl and OH; and where said C₃-C₇ cycloalkyl and heterocycloalkyl are optionally substituted with one to three C₁-C₃ alkyl groups;
each Rd is independently optionally substituted C₁-C₃ alkyl, where said C₁-C₃ alkyl is optionally substituted with one to three substituents each independently selected from the group consisting of: C₃-C₆ cycloalkyl; phenyl optionally substituted with one to three substituents each independently selected from the group consisting of: halo, C₁-C₆ alkyl, and C₃-C₆ cycloalkyl; and heteroaryl optionally substituted with one to three substituents each independently selected from the group consisting of: halo, C₁-C₆ alkyl, and C₃-C₆ cycloalkyl;
each Re is independently selected from the group consisting of: optionally substituted C₁-C₆ alkyl, optionally substituted phenyl, optionally substituted heteroaryl, optionally substituted C₅-C₇ cycloalkyl, and optionally substituted heterocycloalkyl, where said C₁-C₆ alkyl is optionally substituted with one substituent selected from the group consisting of: ORc, trifluoromethyl, phenyl, heteroaryl, heterocycloalkyl optionally substituted with ORc or heterocycloalkyl, and NRaRb; where said phenyl and heteroaryl are optionally substituted with one to three substituents each independently selected from the group consisting of: halo, CN, C₁-C₆ alkyl, C₁-C₆ haloalkyl, N(Rb)C(O)Ra, and ORf; and where said C₅-C₇ cycloalkyl and heterocycloalkyl are optionally substituted with one to three substituents each independently selected from the group consisting of: halo, C₁-C₆ alkyl optionally substituted with ORc and C₃-C₆ cycloalkyl;
each Rf is independently selected from the group consisting of: H and C₁-C₆ alkyl;
each Rg is independently selected from the group consisting of: H, C₁-C₆ alkyl, C₃-C₆ cycloalkyl, heteroaryl, and phenyl;
each Rh is independently selected from the group consisting of: H and C₁-C₆ alkyl optionally substituted with one phenyl group;
each Ri is independently selected from the group consisting of: H, C₁-C₆ alkyl, C₁-C₆ haloalkyl, and phenyl; and
Rj is optionally substituted aryl, optionally substituted heteroaryl, optionally substituted C₃-C₆ cycloalkyl, or optionally substituted heterocycloalkyl,
where said aryl and heteroaryl are optionally substituted with one to three substituents each independently selected from the group consisting of: —ORf, nitro, cyano, CF₃, C₁-C₆ alkyl, C(O)Rf, COORf, —NRfRg, —NHC(O)Rf, —C(O)NRfRg, —S(O)₂Rf, —NHS(O)₂Rf, and —S(O)₂NRfRg; and where said C₃-C₆ cycloalkyl and heterocycloalkyl are optionally substituted with one to three substituents each independently selected from the group consisting of: —OH, oxo, C₁-C₆ alkyl, and C₁-C₆ haloalkyl; or a pharmaceutically acceptable salt thereof.

2. A compound according to claim 1 wherein R3 is H or a pharmaceutically acceptable salt thereof.

3. A compound according to claim 2 wherein R2 is optionally substituted C₁-C₆ alkyl or a pharmaceutically acceptable salt thereof.

4. A compound according to claim 3 wherein R2 is ethyl or a pharmaceutically acceptable salt thereof.

5. A compound according to claim 4 wherein X is O or S; or a pharmaceutically acceptable salt thereof.

6. A compound according to claim 4 wherein X is S(O)₂ or a pharmaceutically acceptable salt thereof.

7. A compound according to claim 4 wherein X is OC(O)O or a pharmaceutically acceptable salt thereof.

8. A compound according to claim 4 wherein X is N(Rf) or a pharmaceutically acceptable salt thereof.

9. A compound according to claim 4 wherein R1 is H, optionally substituted C₁-C₈ alkyl, C₁-C₆ haloalkyl, optionally substituted phenyl, optionally substituted —C₁-C₃ alkylene-phenyl, and optionally substituted C₁-C₃ alkylene-naphthyl,
where said C₁-C₈ alkyl is optionally substituted with one substituent selected from the group consisting of: cyano, —NRfRf, —C(O)NRfRf, C₃-C₆ cycloalkyl, and C₁-C₆ alkoxy optionally substituted with one phenyl group;
where said phenyl, —C₁-C₃ alkylene-phenyl, naphthyl, and —C₁-C₃ alkylene-naphthyl, are each optionally substituted with one to three substituents each independently selected from the group consisting of: halo, —CN, —C(O)NRaRb, —SO₂Ri, —NRaRb, —ORc, —C(O)ORa, C₁-C₆ alkyl, C₁-C₆ alkyl substituted with one to three substituents independently selected from the group consisting of: —NRaRb, C₃-C₆ cycloalkyl, phenyl, —ORc, and C₁-C₆ haloalkyl; or a pharmaceutically acceptable salt thereof.

10. A compound according to claim 1 selected from the group consisting of:
3-[1-(ethylsulfonyl)-4-piperidinyl]-5-hydroxy-1H-indole-7-carboxamide;
5-[(cyclopropyl methyl)oxy]-3-[1-(ethylsulfonyl)-4-piperidinyl]-1H-indole-7-carboxamide;
3-[1-(ethylsulfonyl)-4-piperidinyl]-5-(pentyloxy)-1H-indole-7-carboxamide;
3-[1-(ethylsulfonyl)-4-piperidinyl]-5-(octyloxy)-1H-indole-7-carboxamide;
3-[1-(ethylsulfonyl)-4-piperidinyl]-5-(heptyloxy)-1H-indole-7-carboxamide;
3-[1-(ethylsulfonyl)-4-piperidinyl]-5-[(2-phenylethyl)oxy]-1H-indole-7-carboxamide;
3-[1-(ethylsulfonyl)-4-piperidinyl]-5-[(3-phenylpropyl)oxy]-1H-indole-7-carboxamide;
5-[(2-chloroethyl)oxy]-3-[1-(ethylsulfonyl)-4-piperidinyl]-1H-indole-7-carboxamide;
3-[1-(ethylsulfonyl)-4-piperidinyl]-5-({4-[(phenylmethyl)oxy]butyl}oxy)-1H-indole-7-carboxamide;
3-[1-(ethylsulfonyl)-4-piperidinyl]-5-({2-[(phenylmethyl)oxy]ethyl}oxy)-1H-indole-7-carboxamide;
5-[(3-cyanopropyl)oxy]-3-[1-(ethylsulfonyl)-4-piperidinyl]-1H-indole-7-carboxamide;
5-[(4-amino-4-oxobutyl)oxy]-3-[1-(ethylsulfonyl)-4-piperidinyl]-1H-indole-7-carboxamide;
3-[1-(ethylsulfonyl)-4-piperidinyl]-5-[(phenylmethyl)oxy]-1H-indole-7-carboxamide; and
3-[1-(ethylsulfonyl)-4-piperidinyl]-5-[(phenylmethyl)oxy]-1H-indole-7-carboxamide; or a pharmaceutically acceptable salt thereof.

11. A compound according to claim 1 selected from the group consisting of:
5-{[(3,4-difluorophenyl)methyl]oxy}-3-[1-(ethylsulfonyl)-4-piperidinyl]-1H-indole-7-carboxamide;
5-{[(3-chlorophenyl)methyl]oxy}-3-[1-(ethylsulfonyl)-4-piperidinyl]-1H-indole-7-carboxamide;
methyl-4-[({7-(aminocarbonyl)-3-[1-(ethylsulfonyl)-4-piperidinyl]-1H-indol-5-yl}oxy)methyl]benzoate;
3-[1-(ethylsulfonyl)-4-piperidinyl]-5-{[(4-fluorophenyl)methyl]oxy}-1H-indole-7-carboxamide;
5-{[(3-cyanophenyl)methyl]oxy}-3-[1-(ethylsulfonyl)-4-piperidinyl]-1H-indole-7-carboxamide;
3-[1-(ethylsulfonyl)-4-piperidinyl]-5-[({2-[(phenylsulfonyl)methyl]phenyl}methyl)oxy]-1H-indole-7-carboxamide;
5-{[(2-cyanophenyl)methyl]oxy}-3-[1-(ethylsulfonyl)-4-piperidinyl]-1H-indole-7-carboxamide;
3-[1-(ethylsulfonyl)-4-piperidinyl]-5-[(2-naphthalenylmethyl)oxy]-1H-indole-7-carboxamide;
3-[1-(ethylsulfonyl)-4-piperidinyl]-5-[({3-[(trifluoromethyl)oxy]phenyl}methyl)oxy]-1H-indole-7-carboxamide;
3-[1-(ethylsulfonyl)-4-piperidinyl]-5-({[2-fluoro-4-(trifluoromethyl)phenyl]methyl}oxy)-1H-indole-7-carboxamide;
5-{[(3,5-difluorophenyl)methyl]oxy}-3-[1-(ethylsulfonyl)-4-piperidinyl]-1H-indole-7-carboxamide;
5-[({3-[(difluoromethyl)oxy]phenyl}methyl)oxy]-3-[1-(ethylsulfonyl)-4-piperidinyl]-1H-indole-7-carboxamide;
5-{[(3,4-dichlorophenyl)methyl]oxy}-3-[1-(ethylsulfonyl)-4-piperidinyl]-1H-indole-7-carboxamide;
5-{[(4-chlorophenyl)methyl]oxy}-3-[1-(ethylsulfonyl)-4-piperidinyl]-1H-indole-7-carboxamide;
3-[1-(ethylsulfonyl)-4-piperidinyl]-5-[(4-methylphenyl)oxy]-1H-indole-7-carboxamide;
3-[1-(ethylsulfonyl)-4-piperidinyl]-5-{[4-(methyloxy)phenyl]oxy}-1H-indole-7-carboxamide;
5-{[3-(diethylamino)phenyl]oxy}-3-[1-(ethylsulfonyl)-4-piperidinyl]-1H-indole-7-carboxamide;
3-[1-(ethylsulfonyl)-4-piperidinyl]-5-[(4-fluorophenyl)oxy]-1H-indole-7-carboxamide;
3-[1-(ethylsulfonyl)-4-piperidinyl]-5-[(2-methylpropyl)amino]-1H-indole-7-carboxamide;
3-[1-(ethylsulfonyl)-4-piperidinyl]-5-[methyl(phenyl)amino]-1H-indole-7-carboxamide;
3-[1-(ethylsulfonyl)piperidin-4-yl]-5-(phenylthio)-1H-indole-7-carboxamide;
5-[(4-chlorophenyl)thio]-3-[1-(ethylsulfonyl)-4-piperidinyl]-1H-indole-7-carboxamide;
5-[(2-chlorophenyl)thio]-3-[1-(ethylsulfonyl)-4-piperidinyl]-1H-indole-7-carboxamide;
3-[1-(ethylsulfonyl)-4-piperidinyl]-5-[(4-methylphenyl)thio]-1H-indole-7-carboxamide;
3-[1-(ethylsulfonyl)-4-piperidinyl]-5-[(4-fluorophenyl)thio]-1H-indole-7-carboxamide;
3-[1-(ethylsulfonyl)-4-piperidinyl]-5-[(3-fluorophenyl)thio]-1H-indole-7-carboxamide;
3-[1-(ethylsulfonyl)-4-piperidinyl]-5-[(3-fluorophenyl)thio]-1H-indole-7-carboxamide;
3-[1-(ethylsulfonyl)-4-piperidinyl]-5-[(2-fluorophenyl)thio]-1H-indole-7-carboxamide;
3-[1-(ethylsulfonyl)-4-piperidinyl]-5-[(3-methylphenyl)thio]-1H-indole-7-carboxamide;
5-{[2-(diethylamino)ethyl]thio}-3-[1-(ethylsulfonyl)-4-piperidinyl]-1H-indole-7-carboxamide;
5-[(2,4-dichlorophenyl)thio]-3-[1-(ethylsulfonyl)-4-piperidinyl]-1H-indole-7-carboxamide;
3-[1-(ethylsulfonyl)-4-piperidinyl]-5-[(2-methylpropyl)thio]-1H-indole-7-carboxamide;
5-{[4-(acetylamino)phenyl]thio}-3-[1-(ethylsulfonyl)-4-piperidinyl]-1H-indole-7-carboxamide;
3-[1-(ethylsulfonyl)-4-piperidinyl]-5-[(4-methylphenyl)sulfonyl]-1H-indole-7-carboxamide;
3-[1-(ethylsulfonyl)-4-piperidinyl]-5-[(3-fluorophenyl)sulfonyl]-1H-indole-7-carboxamide; and
5-{[4-(acetylamino)phenyl]sulfonyl}-3-[1-(ethylsulfonyl)-4-piperidinyl]-1H-indole-7-carboxamide; or a pharmaceutically acceptable salt thereof.

12. A pharmaceutical composition comprising a compound according to claim 1, or a pharmaceutically acceptable salt thereof, and one or more of pharmaceutically acceptable carriers.

13. A method of treating a disorder mediated by inappropriate IKK2 activity comprising administering a safe and effective amount of a compound according to claim 1, or a pharmaceutically acceptable salt thereof, to a patient in need thereof.

14. A method according to claim 13 wherein the disorder mediated by inappropriate IKK2 activity is an inflammatory or tissue repair disorder.

15. A method according to claim 13 wherein the disorder mediated by inappropriate IKK2 activity is an autoimmune disease.

16. A method according to claim 15 wherein the autoimmune disease is systemic lupus eythematosus, multiple sclerosis, psoriatic arthritis, or alkylosing spondylitis.

17. A method according to claim 13 wherein the disorder mediated by inappropriate IKK2 activity is selected from the group consisting of: rheumatoid arthritis, inflammatory bowel disease, asthma, COPD (chronic obstructive pulmonary disease) osteoarthritis, osteoporosis, psoriasis, atopic dermatitis, ultraviolet radiation (UV)-induced skin damage, systemic lupus eythematosus, multiple sclerosis, psoriatic arthritis, alkylosing spondylitis, tissue rejection, organ rejection, Alzheimer's disease, stroke, atherosclerosis, restonosis, diabetes, glomerulonephritis, Hodgkins disease, cachexia, inflammation associated with infection and certain viral infections, including acquired immune deficiency syndrome (AIDS), adult respiratory distress syndrome, and Ataxia Telangiestasia.

18. A method according to claim 17 wherein the disorder mediated by inappropriate IKK2 activity is rheumatoid arthritis, asthma or COPD.

19. A method according to claim 18 wherein the disorder mediated by inappropriate IKK2 activity is rheumatoid arthritis.

20. A method according to claim 18 wherein the disorder mediated by inappropriate IKK2 activity is asthma.

21. A method according to claim 18 wherein the disorder mediated by inappropriate IKK2 activity is COPD.

22. A method according to claim 17 wherein the disorder mediated by inappropriate IKK2 activity is selected from the group consisting of: Alzheimer's disease, stroke atherosclerosis, restenosis, diabetes, glomerulonephritis, osteoarthritis, osteoporosis, and Ataxia Telangiestasia.

23. A method according to claim 13 wherein the disorder mediated by inappropriate IKK2 activity is cancer or cachexia.
